(19) United States
(12) Patent Application Publication
(10) Pub. No.: US 2018/0188264 A1
(43) Pub. Date: Jul. 5, 2018

(54) SYSTEMS, METHODS AND COMPOSITIONS FOR SIMULTANEOUS DETECTION OF RNA AND PROTEIN BY MASS SPECTROMETRY

(71) Applicant: Fluidigm Canada Inc., Markham (CA)

(72) Inventor: Olga Ornatsky, Richmond Hill (CA)

(21) Appl. No.: 15/740,527

(22) PCT Filed: Jun. 29, 2016
(86) PCT No.: PCT/US2016/040095

§ 371 Date: Dec. 28, 2017

Related U.S. Application Data
(60) Provisional application No. 62/186,355, filed on Jun. 29, 2015.

(51) Int. Cl. G01N 33/68; C12Q 1/6872; C12Q 1/6818

(52) U.S. Cl. CPC G01N 33/6851; C12Q 1/6872; C07H 21/02; C12Q 2565/627; C12Q 1/6818

(57) ABSTRACT

In certain embodiments, a method of detection includes providing cells labeled with a first element tag bound to a target RNA sequence and a second element tag bound to a target protein. The method may further include ionizing a cell, or a portion thereof, to produce an ionized first and second element tag, and simultaneously detecting the ionized first and second element tag. The ionization may be performed by an inductively coupled plasma (ICP) torch. The method may further comprise nebulizing the cells in suspension prior to ionizing. In certain embodiments, the ionized first and second element tag may be detected by time-of-flight mass spectrometry. Additional methods, and systems and reagents for simultaneous detection of RNA and protein by mass spectrometry are also described.

### CROSS-REFERENCE TO RELATED APPLICATIONS

This application claims benefit of U.S. provisional application No. 62/186,355 filed Jun. 29, 2015, the entire contents of which are incorporated herein by reference.

### FIELD OF THE INVENTION

This invention relates to methods and compositions for simultaneously detecting RNA and protein in a biological sample by mass spectrometry.

### BACKGROUND OF THE INVENTION

In the life sciences, RNA research is seen as one of the fastest growing fields. It is expected to influence our understanding of many diseases, resulting in diverse biotechnological applications with the potential to impact healthcare. RNA research (Transcriptomics) investigates the biogenesis, structure and function of different types of RNA.

RNA research investigates the biogenesis, structure and function of different types of RNA and focuses on the mechanisms and regulation of messenger RNA translation, splicing, polyadenylation, turnover, intracellular localization and RNA interference, as well as mRNA transport, processing and control by small non-coding RNAs.

### SUMMARY

In certain embodiments, a method of detection includes providing cells labeled with a first element tag bound to a target RNA sequence and a second element tag bound to a target protein. The method may further include ionizing a cell, or a portion thereof, to produce an ionized first and second element tag, and simultaneously detecting the ionized first and second element tag. The ionization may be performed by an inductively coupled plasma (ICP) torch. The method may further comprise nebulizing the cells in suspension prior to ionizing. In certain embodiments, the ionized first and second element tag may be detected by time-of-flight mass spectrometry. Additional methods, and systems and reagents for simultaneous detection of RNA and protein by mass spectrometry are also described.

### BRIEF DESCRIPTION OF THE FIGURES

The figures described below are for illustration purposes only and are not intended to limit the scope of this disclosure.

FIG. 1 provides an exemplary hybridization scheme to hybridize a target RNA and provide an amplified mass signal.

FIG. 2 provides exemplary conjugation chemistries for coupling a element tag to an oligonucleotide.

FIG. 3 provides an FPLC graph showing the 214 nm and 260 nm absorbance across elution time. The collected element-tagged oligonucleotide is the left peak, while the unreacted oligonucleotide is the right peak.

FIG. 4 provides a mass cytometry analysis of IFNy protein, IFNy mRNA and CD4 mRNA expression in human peripheral blood mononuclear cells (PBMCs) under unstimulated (A) and PMA/ionomycin stimulation (B) conditions.

### DETAILED DESCRIPTION

In certain embodiments, a method of detection includes providing cells labeled with a first element tag bound to a target RNA sequence and a second element tag bound to a target protein. The method may further include ionizing a cell, or a portion thereof, to produce ionized first and second element tags, and simultaneously detecting the ionized first and second element tags. The ionization may be performed by an inductively coupled plasma (ICP) torch. The method may further comprise nebulizing the cells in suspension prior to ionizing. In certain embodiments, the ionized first and second element tag may be detected by time-of-flight mass spectrometry. Additional methods, and systems and reagents for simultaneous detection of RNA and protein by mass spectrometry are also described.

### Definitions

Terms used in the claims and specification are defined as set forth below unless otherwise specified. These terms are defined specifically for clarity, but all of the definitions are consistent with how a skilled artisan would understand these terms.

The term “simultaneous” in the context of detecting both RNA (e.g., one or more RNA sequences) and protein (e.g., one or more proteins), means detection of elemental ions corresponding to the presence of both the RNA and protein at the same time, e.g., within a fraction of a second of one another. Often, multiple mass spectra will be taken of a single cell, each providing the abundance of both the elemental ions associated with the RNA target and elemental ions associated with the protein target. In the context of hybridization, “simultaneous” means hybridizing to different sequences at the same time.

“Element tag” or “tag” is a chemical moiety which includes an element, or multiple elements, having one or many isotopes (referred to as “tag atoms”) attached to a supporting molecular structure, or that is capable of binding said element(s) or isotope(s). The element tag can also comprise the means of attaching the element tag to a molecule of interest or target molecule (for example, an analyte). Different element tags may be distinguished on the basis of the elemental composition of the tags. An element tag can contain many copies of a given isotope and can have a reproducible copy number of each isotope in each tag. An element tag is functionally distinguishable from other element tags in the same sample because its elemental or isotopic composition is different from that of the other tags.

A “polymer” is a substance composed of molecules characterized by the multiple repetitions of one or more species of atoms or groups of atoms (constitutional units) linked to each other in amounts sufficient to provide a set of properties that do not vary markedly with the addition or removal of one or a few constitutional units. (IUPAC definition, see E. S. White, J. Chem. Inf. Comput. Sci. 1997, 37, 171-192). A polymer molecule can be thought of in terms of its backbone, the connected link of atoms that span the length of the molecule, and the pendant groups, attached to the backbone portion of each constituent unit. The pendant groups are often chemically and functionally different from the backbone chain. Pendant groups that have a high affinity for metal ions can act as chelating groups or ligands for those ions. and polysaccharides.

A “linear polymer” is a polymer characterized by a linear sequence of constituent units. A “block copolymer” is a linear polymer with sequences of constituent units of a common type, joined to sequences of constituent units of a different type. A “branched polymer” is a polymer in which additional polymer chains (the branches) issue from the backbone of the polymer. One commonly refers to the longest linear sequence as the “main chain”. A branched polymer in which the chemical composition of the constituent units of the branch chains is different than those of the main chain is called a “graft copolymer”.

A “metal tagged polymer” (also a “polymeric metal tag carrier”, or “metal-polymer conjugate”, or “chelate-derivatized polymer”) is a variety of the element tag which has a polymer backbone bearing at least one pendant chelating group with metal atoms attached to them. These metal tagged polymers can be, but are not limited to, linear, star, branched, or hyperbranched homopolymers or copolymers as well as block or graft copolymers.

A “metal binding pendant group” is a pendant group on the polymer that is capable of binding a metal or an isotope of a metal. It can also be referred to as a ligand.

“Chelation” is the process of binding of a ligand, the chelant, chelator or chelating agent, to a metal ion, forming a metal complex, the chelate. In contrast to the simple monodentate ligands like H₂O or NH₃, the polydentate chelators form multiple bonds with the metal ion.

“Transition element” means an element having one of the following atomic numbers: 21-30, 39-48, 57-80 and 89-92. Transition elements include the rare earth metals, lanthanides and noble metals.

“Lanthanides” are the transition metals with atomic numbers from 57 to 71 including La, Ce, Pr, Nd, Pm, Sm, Eu, Gd, Tb, Dy, Ho, Er, Tm, Yb, Lu.

“Metal” means an element having one of the following atomic numbers 3, 4, 11-13, 19-33, 37-52, 55-84, 87-102.

An “attachment (linker) group” or “linker” is a portion of a molecule that is used to couple (conjugate) two different molecules or polymers, two subunits of a molecule, or a molecule to a substrate, for example an affinity agent.

“ICP-MS” is the Inductively Coupled Plasma Mass Spectrometer—a sensitive mass spectrometry based elemental analyzer. Different ICP-MS configurations are primarily distinguished by the mass selecting technique employed and can be, for example the quadrupole or time-of-flight (ICP-TOF) or magnetic sector (high resolution ICP-MS). There are many commercially available ICP-MS models having a wide spectrum of configurations, capabilities and modifications. An examplary ICP-MS is the CyTOF® Mass Cytometer available from Fluidigm Canada.

“Affinity reagent”, as used herein, is a non-oligonucleotide biomolecule capable of tightly binding to a target molecule or analyte. For example, an antibody is an affinity reagent that recognizes and binds with high affinity to a specific antigen (e.g., on a protein).

A “nucleic acid target” or “target nucleic acid” refers to a nucleic acid (e.g., RNA, DNA), or optionally a region thereof, that is to be detected.

A “polynucleotide sequence” or “nucleotide sequence” is a polymer of nucleotides (an oligonucleotide, a DNA, a nucleic acid, etc.) or a character string representing a nucleotide polymer, depending on context. From any specified polynucleotide sequence, either the given nucleic acid or the complementary polynucleotide sequence (e.g., the complementary nucleic acid) can be determined.

The term “gene” is used broadly to refer to any nucleic acid associated with a biological function. Genes typically include coding sequences and/or the regulatory sequences required for expression of such coding sequences. The term gene can apply to a specific genomic sequence, as well as to a cDNA or an mRNA encoded by that genomic sequence.

The term “antibody” is used herein in the broadest sense and covers fully assembled antibodies, antibody fragments which retain the ability to specifically bind to the antigen (e.g., Fab, F(ab′)2, Fv, and other fragments), single chain antibodies, diabodies, antibody chimeras, hybrid antibodies, bispecific antibodies, humanized antibodies, and the like. The term “antibody” covers both polyclonal and monoclonal antibodies.

The term “biological sample” or “tissue sample” as used herein refers to a sample obtained from a biological subject, including sample of biological tissue or fluid origin, obtained, reached, or collected in vivo or in situ. A biological sample also includes samples from a region of a biological subject containing precancerous or cancer cells or tissues. Such samples can be, but are not limited to, organs, tissues, fractions and cells isolated from a mammal. Exemplary biological samples include but are not limited to cell lysate, a cell culture, a cell line, a tissue, an organ, an organelle, a biological fluid, and the like. Exemplary biological samples include but are not limited to a skin sample, tissue biopsies, and the like.

The term “label probe” or “element-tagged oligonucleotide” refers to an oligonucleotide having one or more elemental tags, and that binds to a target nucleic acid, directly or indirectly, and enables the target to be detected, e.g., by a mass spectrometer.

A “capture probe” or “target hybridization oligonucleotide” is a polynucleotide that is capable of hybridizing to a target nucleic acid and capturing a label probe to that target nucleic acid. The target probe can hybridize directly to the label probe, or it can hybridize to one or more nucleic acids that in turn hybridize to the label probe; for example, the target probe can hybridize to an amplifier or a preamplifier. The target probe thus includes a first polynucleotide sequence that is complementary to a polynucleotide sequence of the target nucleic acid and a second polynucleotide sequence that is complementary to a polynucleotide sequence of the label probe, amplifier, preamplifier, or the like. The target probe is preferably single-stranded.

An “amplifier” or in some cases “amplification oligonucleotide” is a molecule, typically a polynucleotide, that is capable of hybridizing to multiple label probes. Typically, the amplifier hybridizes to multiple identical label probes. The amplifier also hybridizes to at least one target probe or nucleic acid bound to a target probe. For example, the amplifier can hybridize to at least one target probe and to a plurality of label probes, or to a preamplifier and a plurality of label probes. The amplifier can be, e.g., a linear, forked, comb-like, or branched nucleic acid. As noted for all polynucleotides, the amplifier can include modified nucleotides and/or nonstandard internucleotide linkages as well as standard deoxyribonucleotides, ribonucleotides, and/or phosphodiester bonds. Suitable amplifiers are described, for example, in U.S. Pat. No. 5,635,352, U.S. Pat. No. 5,124,246, U.S. Pat. No. 5,710,264, and U.S. Pat. No. 5,849,481, incorporated herein by reference.

A “preamplifier” or in some cases “amplification oligonucleotide” is a molecule, typically a polynucleotide, that serves as an intermediate between one or more target probes and amplifiers. Typically, the preamplifier hybridizes simultaneously to one or more target probes and to a plurality of amplifiers. Exemplary preamplifiers are described, for example, in U.S. Pat. No. 5,635,352 and U.S. Pat. No. 5,681,697, incorporated herein by reference.

The term “ISH” or “in situ hybridization” refers to a type of hybridization that uses a labeled complementary DNA or RNA strand (i.e., probe) to localize a specific DNA or RNA sequence in a portion or section of tissue (in situ). The probe types are double stranded DNA (dsDNA), single stranded DNA (ssDNA), single stranded complimentary RNA (sscRNA), messenger RNA (mRNA), micro RNA (miRNA), and synthetic oligonucleotides.

The term “oligonucleotide,” as used herein, hybridizes to a nucleic acid (e.g., RNA) in a sequence specific manner and may comprise naturally occurring nucleotides and/or non-naturally occurring nucleotide analogs. An oligonucleotide will generally contain phosphodiester bonds, although in some cases, nucleic acid analogs are included that may have alternate backbones, comprising, for example and without limitation, phosphoramide (Beaucage et al., Tetrahedron 49(10): 1925 (1993) and references therein; Letsinger, J. Org. Chem. 35:3800 (1970); Sprinzl et al., Eur. J. Biochem. 81:579 (1977); Letsinger et al., Nucl. Acids Res. 14:3487 (1986); Sawai et al, Chem. Lett. 805 (1984), Letsinger et al., J. Am. Chem. Soc. 110:4470 (1988); and Pauwels et al., Chemica Scripta 26:141 91986)), phosphorothioate (Mag et al., Nucleic Acids Res. 19:1437 (1991); and U.S. Pat. No. 5,644,048), phosphorodithioate (Briu et al., J. Am. Chem. Soc. 111:2321 (1989), O-methylphophoroamidite linkages (see Eckstein, Oligonucleotides and Analogues: A Practical Approach, Oxford University Press), and peptide nucleic acid backbones and linkages (see Egholm, J. Am. Chem. Soc. 114:1895 (1992); Meier et al., Chem. Int. Ed. Engl. 31:1008 (1992); Nielsen, Nature, 365:566 (1993); Carlsson et al., Nature 380:207 (1996), all of which are incorporated by reference). Other analog nucleic acids include those with bicyclic structures including locked nucleic acids, Koshkin et al., J. Am. Chem. Soc. 120:13252 3 (1998); positive backbones (Denpcy et al., Proc. Natl. Acad. Sci. USA 92:6097 (1995); non-ionic backbones (U.S. Pat. Nos. 5,386,023, 5,637,684, 5,602,240, 5,216,141 and 4,469,863; Kiedrowshi et al., Angew. Chem. Intl. Ed. English 30:423 (1991); Letsinger et al., J. Am. Chem. Soc. 110:4470 (1988); Letsinger et al., Nucleoside & Nucleotide 13:1597 (1994); Chapters 2 and 3, ASC Symposium Series 580, “Carbohydrate Modifications in Antisense Research”, Ed. Y. S. Sanghui and P. Dan Cook; Mesmaeker et al., Bioorganic & Medicinal Chem. Lett. 4:395 (1994); Jeffs et al., J. Biomolecular NMR 34:17 (1994); Tetrahedron Lett. 37:743 (1996)) and non-ribose backbones, including those described in U.S. Pat. Nos. 5,235,033 and 5,034,506, and Chapters 6 and 7, ASC Symposium Series 580, “Carbohydrate Modifications in Antisense Research”, Ed. Y. S. Sanghui and P. Dan Cook. Nucleic acids containing one or more carbocyclic sugars are also included within the definition of nucleic acids (see Jenkins et al., Chem. Soc. Rev. (1995) pp 169 176).

### Element Tags

The overall requirements for an element tag are less stringent than those for a fluorescent tag, since the chemical nature of an element is not important for its detection by elemental analysis. The tag should contain a reproducible and, preferably, large number of atoms of a given element or isotope composition. The tag can comprise one element or isotope, or consist of a composition of more than one element or isotope. It can also include a natural mixture of isotopes. Further, it is possible that the element tag can comprise one pendant group comprising a certain metal or isotope and a second pendant group comprising another metal or isotope. Reproducibility in the number of identical atoms incorporated is a basis for quantitative analysis, and an increase in the number of those atoms improves the sensitivity linearly.

In certain embodiments, element tags may not be distinguishable from one another by colormetric measures, such as fluorescence and/or absorbance. Further, element tags may not be detectable by fluorescence (e.g., may not exhibit fluorescence above the background fluorescence present in a cell).

An aspect of the invention is to provide an element tag comprising a polymer, wherein the polymer comprises at least one metal-binding pendant group that comprises at least one metal atom or is capable of binding at least one metal atom. The element tag can further comprise a functional group that allows the polymer to be attached to one of a linker, a spacer, or a biomolecule. The element tag can be water soluble. It can also be negatively charged. The number of metal-binding pendant groups capable of binding at least one metal atom can be between approximately 1 and 1000, and most typically between approximately 10 and 250. At least one metal atom can be bound to at least one of the metal-binding pendant groups. The polymer can have a degree of polymerization of between approximately 1 and 1000, and most typically between 10 and 250. The element tag polymer may therefore have between 10 and 1000 metal atoms, 10 and 100 metal atoms, more than 10 metal atoms, more than 20 metal atoms, more than 50 metal atoms, more than 100 metal atoms, or more than 1000 metal atoms.

The polymer can be selected from the group consisting of linear polymers, copolymers, branched polymers, graft copolymers, block polymers, star polymers, and hyperbranched polymers. The backbone of the polymer can be derived from substituted polyacrylamide, polymethacrylate, or polymethacrylamide and can be a substituted derivative of a homopolymer or copolymer of acrylamides, methacrylamides, acrylate esters, methacrylate esters, acrylic acid or methacrylic acid.

The metal-binding pendant group can be attached to the polymer through an ester or through an amide. The functional group can be a thiol-reactive group. The metal atom can be a transition element or an isotope thereof, or a lanthanide or an isotope of a lanthanide. The element tag can further comprise a linker attached to the functional group of the polymer, wherein the linker is capable of covalent attachment to a biomolecule. The element tag can further comprise a spacer attached to the linker, wherein the spacer is capable of attachment to a biomolecule. The spacer can be a polyethylene glycol (PEG) spacer. The spacer can comprise a functional group that is capable of binding the spacer to the polymer via a spacer-reactive functional group on the polymer. Further, the spacer can contain a functional group that is capable of binding a linker to the spacer.

The element tag described above, can be covalently attached to a biomolecule. The biomolecule can be a nucleotide or an oligonucleotide. The biomolecule can also be an affinity reagent, such as an antibody or a fragment thereof. Suitable biomolecules are discussed in greater detail in a separate section.

As examples, two ligand frameworks as functional examples of covalently linked chelates on the polymeric backbone are described. The selection criteria for this embodiment include known syntheses, heptadentate or octadentate coordination to promote kinetic stability against metal ion dissociation, a pendant primary amine functional group for attachment of the chelate to the polymer, and a net charge of −1 for the liganded chelate. Diethylenetriaminepentaacetate (DTPA), an acyclic chelator can be readily derivatized as an amine functionalized ligand. Coupling a monoprotected diamine with the commercially available DTPA anhydride, followed by deprotection provides a candidate ligand to be coupled to the polymeric active ester. The net charge of the compound once complexed to lanthanide is −1. The facile synthesis of this chelator makes it an attractive starting point for optimizing the polymeric backbone with attached chelators.

DTPA ligands are inherently more kinetically labile than the macrocyclic ligand based on the cyclen framework. The macrocyclic nature of the cyclen-based ligands pre-organizes the metal binding site, leading to greater thermodynamic and kinetic stability. These chelates are known to be stable in vivo for days 17. Reaction of commercially available tritertbutylmethylcyclen (Macrocylics) with the readily available homoserine derivative provides an orthogonally protected DOTA derivative. The Fmoc protecting group can be removed to access the amine and make it available to couple with the polymeric backbone. In some instances it may be necessary to employ a spacer between the DOTA chelate and the polymer. A variety of selectively protected amino acids of different lengths is commercially available and can be readily coupled and deprotected to form linkers. The lanthanide complex of this chelate will carry a net −1 charge. Based on functionality, these Ln chelates with the reactive —NH2 group are referred to as (L)-NH2.

The element tag may be functionalized with a reactive group to enable conjugation to a biomolecule, such as an oligonucleotide or a non-oligonulceotide affinity reagent. Reactive groups may include amines, thiols, phosphates, haloacetyl groups, maleimides, and so forth. A full discussion of suitable reactive groups and conjugation chemistries is provided in a later section.

As discussed above, an element tag may comprise any of an number of elements, including metals such as transition metals, metalloids, rare earth metals, and lanthanides. The element tag may be of a specific isotope. Often, a metal may exist in any of a number of isotopes, each of which may be used in a separate element tag. As such, dozens of metals are suitable to the subject invention, any of which may be used in separate element tags to allow for a multiplexed approach.

Suitable element tags, and methods of making such element tags, are discussed in US Patent Publication No. 20080003616, which is incorporated herein by reference.

### Conjugation of Element Tag to Biomolecule

An element tag may be coupled to a biomolecule, such as an oligonucleotide probe or a non-oligonucleotide affinity reagent. As an element tag may include any of a large number of elements distinguishable by mass spectrometry, multiple element tags may be conjugated to a variety of oligonucleotide probes and non-oligonucleotide affinity reagents for use in multiplexed assays.

While exemplary conjugation techniques are discussed herein, it is important to note that a large number of conjugation reagents and techniques are available to one of skill in the art, any of which may be suitable for the subject invention. “Bioconjugate Techniques”, 3rd Edition (2013) by Greg T. Hermanson discusses a number of such techniques, incorporated herein by reference. Conjugation chemistries are also described in US Patent Publication No. 20080003616, which has been incorporated herein by reference.

In certain aspects, the element tag may have a first reactive group and the oligonucleotide or non-oligonucleotide affinity reagent may have a second reactive group. Conjugation may include reacting the first and second reactive groups to form a covalent bond. Alternatively, both the first and second reactive groups may be reacted with a crosslinker, such as a heterofunctional crosslinker.

In amine-based bioconjugation, an amine is reacted with an amine-reactive group such as isothiocyanate, isocyanate, sulfonyl chloride, aldehyde, carbodiimide, acyl azide, anhydride, fluorobenzene, carbonate, N-Hydroxysuccinimide (NSH ester), sulfo-NHS ester, imidoester, epoxide, fluorophenyl ester, and so forth. The element tag may include an amine group and the oligonucleotide or non-oligonucleotide affinity reagent may include an amine-reactive group, or vis-versa. Amines are present on lysine residues and on the N-terminus of a polypeptide chain. As such, non-oligonucleotide affinity reagents such as an antibody may provide multiple amine groups without needing to be modified. An oligonucleotide may be modified to have an amine on at least one of the 3′ position of the 3′ nucleotide or the 5′ position of the 5′ nucleotide.

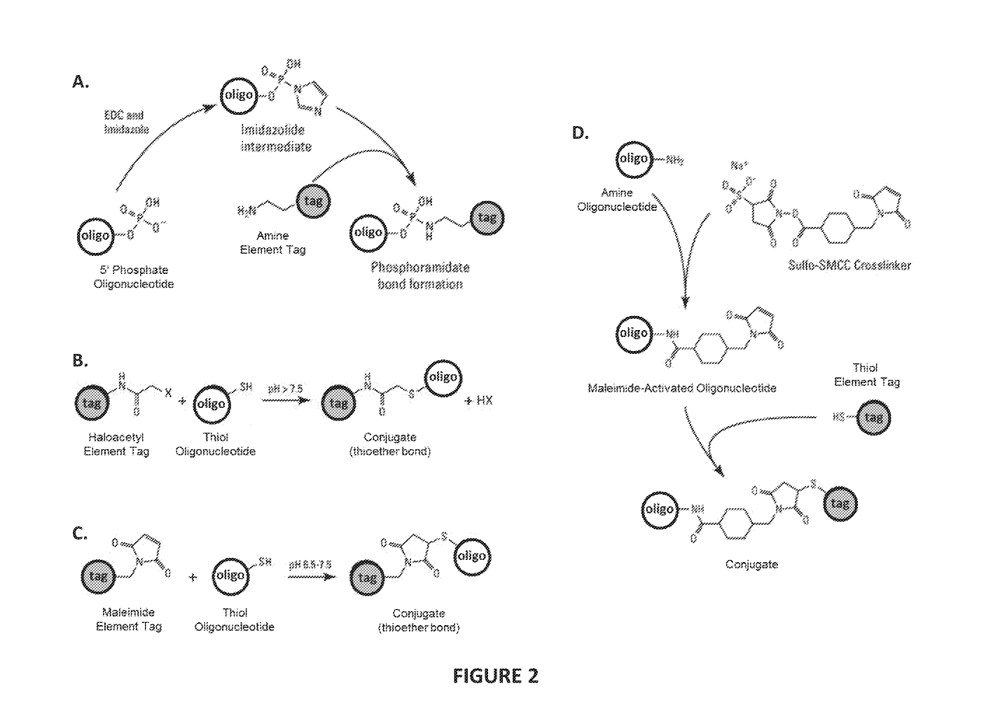

Under certain conditions, a phosphate group can be conjugated to a compound containing a primary amine. As seen in FIG. 2A, an oligonucleotide having a 5′ phosphate group can be reacted in the presence of 1-Ethyl-3-[3-dimethylaminopropyl]carbodiimide (EDC) and 1,3-diazacyclopenta-2,4-diene (imidazole) to form an imidazolide intermediate that can then react with the amine on an element tag. In an alternative example, the oligonucleotide may be functionalized with the the amine and the element tag may be functionalized with the phosphate group.

In thiol-based bioconjugation, a thiol (or sulfhydryl) is reacted with a thiol-reactive group, such as a haloacetamide (e.g., iodoactamide, bromoacetamide, chloroacetamide), maleimide and bismaelimide. FIG. 2B shows a haloacetyl functionalized element tag reacting with a thiol functionalized oligonucleotide to form a thioether bond. In an alternative example, the oligonucleotide may be functionalized with the haloacetyl group and the element tag be functionalized with the thiol. The chemistry shown in FIG. 2B would is suitable for iodoactamide, bromoacetamide, chloroacetamide. FIG. 2C shows a maleimide functionalized element tag reacting with a thiol functionalized oligonucleotide to form a thioether bond. In an alternative example, the oligonucleotide may be functionalized with the maleimide and the element tag be functionalized with the thiol.

An oligonucleotide may be modified to have a thiol on at least one of the 3′ and 5′ positions. A non-oligonucleotide affinity reagent, such as an antibody, may comprise thiols on cysteine residues. While thiols (such as those on cysteine residues) may form disulfide bridges, a reducing environment may be used to cleave the disulfide bond allowing for thiol-based bioconjugation. The chemistry shown in FIG. 2C may therefore be performed in a reducing environment. Reductants such as Tris(2-carboxyethyl)phosphine (TCEP) may provide a suitable reducing environment. An oligonucleotide or non-oligonucleotide affinity reagent providing a thiol may be reacted with an element tag having a thio-reactive group. For example, an oligonucleotide with a thiol group at the 5′ position could be reacted in the presence of TCEP with an element tag having a maleimide group to form an element-tagged oligonucleotide. TCEP is known to be an efficient reductant of alkyl disulfides over a wide range of pH and does not interfere with commonly used sulfhydryl-reactive reagents such as maleimide cross-linkers.

To avoid the slow hydrolysis in water that is typical of maleimide and iodoacetamide groups, a strategy in which the thiol-reactive agent is added to the end of the (L)-bearing polymer just prior to tagging of the affinity reagent is possible. This strategy takes advantage of the “click” chemistry and involves the 1,3-dipolar addition of azides to acetylenes, a reaction that has been shown to occur under mild conditions with quantitative yield. To introduce the acetylene unit on the end of polymers bearing a terminal —NH2 group, they are reacted with an active ester derivative of 4-pentynoic acid. The polymer is then set up for a reaction with a derivative of the form X—R—N3, where R is the spacer and X represents the thiol-reactive group.

In certain aspects of the invention, a crosslinker may be used to link the element tag to an oligonucleotide probe or a non-oligonucleotide affinity reagent. The crosslinker may be heterobifunctional, so as to react with a first reactive group on the element tag and a second reactive group on the biomolecule. For example, the croslinker may be an amine-to-sulfhydryl crosslinker, having both an amine reactive group (such as a sulfo-NHS ester) and a thiol-reactive group (such as a maleimide). An example amine-to-sulfhydryl crosslinker is sulfosuccinimidyl 4-(N-maleimidomethyl)cyclohexane-1-carboxylate (Sulfo-SMCC), which is sold by Life Technologies. In certain embodiments, the cross linker may comprise a spacer, such as polyethylene glycol (PEG) or a linear carbon chain such as a C5-C20 alkyl chain.

In certain aspects, the element tag may be conjugated to a biomolecule (e.g., an oligonucleotide or non-oligonucleotide affinity reagent) non-covalently. For example, biotin-avidin provide a high avidity interaction that is difficult to break. Avidin variants are also suitable for conjugation to biotin, and include deglycosylated forms of avidin such as neuravidin and extravidin, avadin mutants, as well as avidin homologues, such as streptavidin. Avidin may be used in a tetrameric, dimeric or monomeric form. For example, an element tag having biotin may be incubated with a biomolecule having avidin or a variant thereof. Alternatively, an element tag having avidin or a variant thereof may be incubated with a biomolecule having biotin.

Alternative affinity-based conjugations include antigen-antibody pairs. Suitable antigens include haptens such as digoxigenin (DIG), dinitrophenol (DNP), biotin and fluorescin, for which high-affinity antibodies exist.

Oligonucleotides may be modified by a number of methods for use in the above coupling techniques. In addition, modified oligonucleotides are readily available through vendors such as Sigma Aldrich, which offers custom DNA oligonucleotides with amine, phosphate, thiol, biotin, digoxigenin and dinitorphenol modifications at the 3′ and 5′ ends. As such, element-tagged oligonucleotides may be made using any of the above conjugation techniques.

The resulting element tagged biomolecule may be purified by any suitable method, including filtration, chromatography (e.g., HPLC, FPLC, size-exclusion, expanded bed adsorbtion, reversed-phase chromatography, etc.), gel electrophoresis, and so forth.

### Element-Tagged Oligonucleotides

Element-tagged oligonucleotides may be made by any of the conjugations with any of the element tags described in the above sections.

The element-tagged oligonucleotide may comprise a DNA or RNA sequence. The oligonucleotide sequence may be 8 to 200 nucleotides in length, 8 to 100 nucleotides long, 8 to 50 oligonucleotides long, 8 to 30 oligonucleotides long, 8 to 20 oligonucleotides long, 12 to 30 oligonucleotides long, more than 8 nucleotides long, more than 12 nucleotides long, more than 20 nucleotides long, more than 50 nucleotides long, less than 200 nucleotides long, less than 100 nucleotides long, less than 50 nucleotides long, less than 30 nucleotides long, less than 20 nucleotides long, and so forth.

The element-tagged oligonucleotide may specifically hybridize directly to the target nucleic acid. Alternatively, the element-tagged oligonucleotide may hybridize indirectly to the target nucleic acid as described in a later section.

The element tagged oligonucleotide may comprise an element tag linked to the oligonucleotide at the 3′ position of the 3′ nucleotide and/or to the 5′ position of the 5′ nucleotide. The element tag may be conjugated to the oligonucleotide by a biotin bound to an avidin or avidin homologue. In some embodiments, the element tag may be separated from or conjugated to the oligonucleotide, for example, by an amine-to-sulflhydryl crosslinker, thioether bond, secondary amine, sulfide, phsophoramidate bond, n amide bond, polyethylene glycol (PEG) spacer, C5-C20 non-cyclic alkyl spacer, or a combination thereof.

In certain embodiments, the element tag may include a metal atom, as described in the above sections. The elemental tag may be a polymer comprising multiple metal atoms, as described in the above sections.

### Biological Samples and Methods of Preparation

Biological samples of use in the subject detection methods and systems include any cells of interest. Cells may be animal cells, such as mammalian (e.g., human, primate, rodent, rat mouse, etc.), other vertebrate cells, or insect cells.

A biological sample may be isolated from an individual, e.g., from a soft tissue or from a bodily fluid, or from a cell culture that is grown in vitro. A biological sample may comprise cells from a soft tissue such as brain, adrenal gland, skin, lung, spleen, kidney, liver, spleen, lymph node, bone marrow, bladder stomach, small intestine, large intestine or muscle, etc. Bodily fluids include blood, plasma, saliva, mucous, phlegm, cerebral spinal fluid, pleural fluid, tears, lactal duct fluid, lymph, sputum, cerebrospinal fluid, synovial fluid, urine, amniotic fluid, and semen, etc. Biological samples also include cells grown in culture in vitro. A cell may be a cell of a tissue biopsy, scrape or lavage or cells. In certain aspect, cells may be of a tissue section, such as a biopsy or hisological slide of any of the above recited soft tissues. The tissue section may be fixed and/or frozen. In other aspects, cells may be provided in an adherent cell culture, or in a slurry (e.g., suspension).

Alternatively, cells may be of an immortalized cell line, such as 293T, 3T3, 4T1, 721, 9L, A2780, A2780ADR, A2780cis, A172, A20, A253, A431, A-549, ALC, B16, B35, BCP-1, BEAS-2B, bEnd.3, BHK-21, BR 293, BxPC3, C2C12, C3H-10T1/2, C6/36, C6, Cal-27, CGR8, CHO, COR-L23, COR-L23/CPR, COR-L23/5010, COR-L23/R23, COS-7, COV-434, CML T1, CMT, CT26, D17, DH82, DU145, DuCaP, E14Tg2a, EL4, EM2, EM3, EMT6/AR1, EMT6/AR10.0, FM3, H1299, H69, HB54, HB55, HCA2, HEK-293, HeLa, Hepa1c1c7, High Five cells, HL-60, HMEpC, HT-29, HUVEC, Jurkat, J558L cells, JY cells, K562 cells, KBM-7 cells, Ku812, KCL22, KG1, KYO1, LNCap, Ma-Mel 1, 2, 3 . . . 48, MC-38, MCF-7, MCF-10A, MDA-MB-231, MDA-MB-157, MDA-MB-361, MDCK II, MG63, MOR/0.2R, MONO-MAC 6, MRC5, MTD-1A, MyEnd, NCI-H69/CPR, NCI-H69/LX10, NCI-H69/LX20, NCI-H69/LX4, NIH-3T3, NALM-1, NW-145, OPCN/OPCT, Peer, PNT-1A/PNT 2, PTK2, Raji, RBL, RenCa, RIN-5F, RMA/RMAS, S2, Saos-2 cells, Sf21, Sf9, SiHa, SKBR3, SKOV-3, T2, T-47D, T84, U373, U87, U937, VCaP, Vero, WM39, WT-49, X63, YAC-1, and/or YAR cells.

In certain embodiments, the cells may be peripheral blood mononuclear cells or a subset thereof (e.g., T-cells, B-cells, monocytes, dendritic cells, macrophages, NK cells, and so forth). In certain embodiments the cells may be cancer cells of any tissue.

Cells may be cultured prior to hybridization and detection of target RNA and/or protein. In certain embodiments, cells may be cultured in the presence of one or more modulators, such as a phorbol 12-myristate 13-acetate (PMA) and ionomycin, or a cytokine such as CCL1, CCL11, CCL12, CCL13, CCL14, CCL15, CCL16, CCL17, CCL18, CCL19, CCL2, CCL20, CCL21, CCL22, CCL23, CCL24, CCL25, CCL26, CCL27, CCL28, CCL3, CCL5, CCL6, CCL7, CCL8, CCL9, CX3CL1, CX3CR1, CXCL1, CXCL10, CXCL11, CXCL13, CXCL14, CXCL15, CXCL16, CXCL17, CXCL2, CXCL3, CXCL5, CXCL6, CXCL7, CXCL9, Gc-MAF, Granulocyte colony-stimulating factor, Granulocyte macrophage colony-stimulating factor, Hepatocyte growth factor, IL 10 family, IL 17 family, IL1A, IL1B, Inflammasome, Interferome, Interferon, Interferon beta 1a, Interferon beta 1b, Interferon gamma, Interferon type I, Interferon type II, Interferon type III, Interferon-stimulated gene, Interleukin, Interleukin 1 family, Interleukin 1 receptor antagonist, Interleukin 10, Interleukin 12, Interleukin 12 subunit beta, Interleukin 13, Interleukin 16, Interleukin 2, Interleukin 23, Interleukin 23 subunit alpha, Interleukin 34, Interleukin 35, Interleukin 6, Interleukin 7, Interleukin 8, Leukemia inhibitory factor, Leukocyte-promoting factor, Lymphokine, Lymphotoxin, Lymphotoxin alpha, Lymphotoxin beta, Macrophage colony-stimulating factor, Macrophage inflammatory protein, Macrophage-activating factor, Monokine, Myokine, Myonectin, Platelet factor, Tumor necrosis factor alpha, Tumor necrosis factors, Vascular endothelial growth inhibitor, XCL1, XCL2, and/or XCR1. In certain embodiments, cells may be cultured under different conditions and compared using the techniques described herein. In some embodiments, cells may be cultured with a protein transport inhibitor, such as brefeldin A, in order to retain cytokines or chemokines that would otherwise be secreted.

Cells of any of the above-described biological samples may be prepared for analysis of RNA and protein content using the methods described herein. In certain aspects, cells are fixed and permeabilized prior to the hybridization steps described below. Cells may be provided as fixed and/or pemeabilized. Some embodiments of the subject methods include fixing the cells. Cells may be fixed by a crosslinking fixative, such as formaldehyde, glutaraldehyde. Alternatively or in addition, cells may be fixed using a precipitating fixative, such as ethanol, methanol or acetone.

Cells may be permeabilized by a detergent, such as polyethylene glycol (e.g., Triton X-100), Polyoxyethylene (20) sorbitan monolaurate (Tween-20), Saponin (a group of amphipathic glycosides), or chemicals such as methanol or acetone. In certain cases, fixation and permeabilization may be performed with the same reagent or set of reagents. Fixation and permeabilization techniques are discussed by Jamur et al. in “Permeabilization of Cell Membranes” (Methods Mol. Biol., 2010).

### In-Situ Hybridization (ISH) Using Element-Tagged Oligonucleotides

Detection of target nucleic acids in the cell, or “in-situ hybridization” (ISH), has previously been performed using fluorophore-tagged oligonucleotide probes. As discussed herein, mass-tagged oligonucleotides, coupled with ionization and mass spectrometry, can be used to detect target nucleic acids in the cell. Methods of in-situ hybridization are known in the art (see Zenobi et al. “Single-Cell Metabolomics: Analytical and Biological Perspectives,” Science vol. 342, no. 6163, 2013). Hybridization protocols are also described in U.S. Pat. No. 5,225,326 and US Pub. No. 2010/0092972 and 2013/0164750, which are incorporated herein by reference.

Prior to hybridization, cells present in suspension or immobilized on a solid support may be fixed and permeabilized as discussed earlier. Permeabilization may allow a cell to retain target nucleic acids while permitting target hybridization nucleotides, amplification oligonucleotides, and/or element-tagged oligonucleotides to enter the cell. The cell may be washed after any hybridization step, for example, after hybridization of target hybridization oligonucleotides to nucleic acid targets, after hybridization of amplification oligonucleotides, and/or after hybridization of mass-tagged oligonucleotides.

In some embodiments, cells are in suspension for all or most of the steps of the method, for ease of handling. However, the methods are also applicable to cells in solid tissue samples (e.g., tissue sections) and/or cells immobilized on a solid support (e.g., a slide or other surface). Thus, in one class of embodiments, cells can be in suspension in the sample and during the hybridization steps. In other embodiments, the cells are immobilized on a solid support during hybridization.

Target nucleic acids include any nucleic acid of interest and of sufficient abundance in the cell to be detected by the subject methods. Target nucleic acids may be RNAs, of which a plurality of copies exist within the cell. For example, 10 or more, 20 or more, 50 or more, 100 or more, 200 or more, 500 or more, or 1000 or more copies of the target RNA may be present in the cell. A target RNA may be a messenger RNA (mRNA), ribosomal RNA (rRNA), transfer RNA (tRNA), small nuclear RNA (snRNA), small interfering RNA (siRNA), long noncoding RNA (IncRNA), or any other type of RNA known in the art. The target RNA may be 20 nucleotides or longer, 20 nucleotides or longer, 20 nucleotides or longer, 50 nucleotides or longer, 100 nucleotides or longer, 200 nucleotides or longer, 500 nucleotides or longer, 1000 nucleotides or longer, between 20 and 1000 nucleotides, between 20 and 500 nucleotides in length, between 40 and 200 nucleotides in length, and so forth.

In certain embodiments, a mass-tagged oligonucleotide may be hybridized directly to the target nucleic acid sequence. However, hybridization of additional oligonucleotides may allow for improved specificity and/or signal amplification.

In certain embodiments, two or more target hybridization oligonucleotides may be hybridized to proximal regions on the target nucleic acid, and may together provide a site for hybridization of an additional oligonucleotides in the hybridization scheme.

In certain embodiments, the mass tagged oligonucleotide may be hybridized directly to the two or more target hybridization oligonucleotides. In other embodiments, one or more amplification oligonucleotides may be added, simultaneously or in succession, so as to hybridize the two or more target hybridization oligonucleotides and provide multiple hybridization sites to which the element-tagged oligonucleotide can bind. The one or more amplification oligonucleotides, with or without the element-tagged oligonucleotide, may be provided as a multimer capable of hybridizing to the two or more target hybridization oligonucleotides.

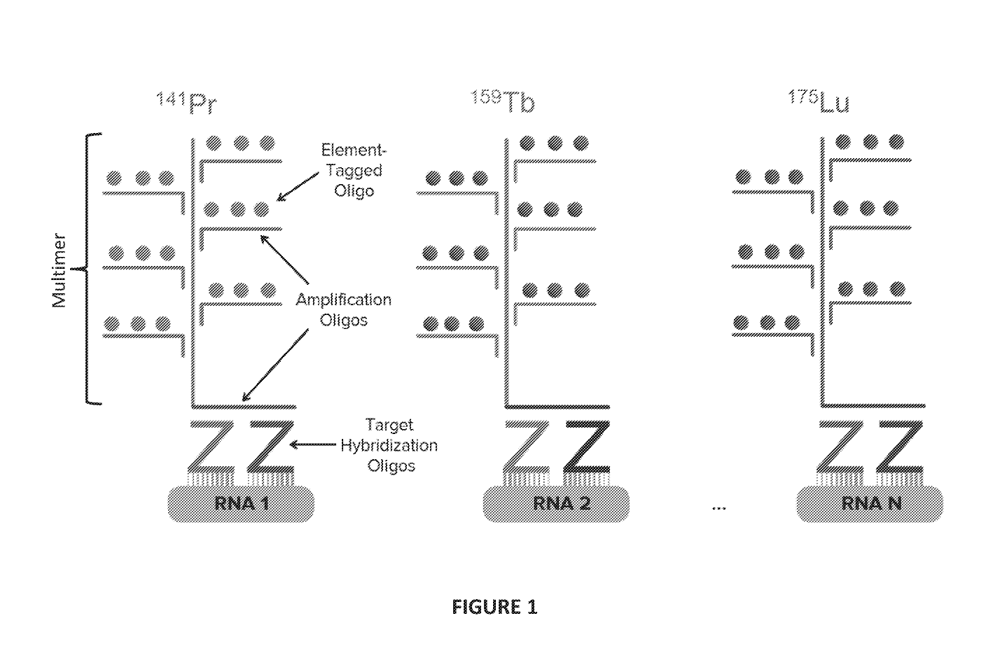

While the use of two or more target hybridization oligonucleotides improves specificity, the use of amplification oligonucleotides increases signal. An example hybridization scheme is shown in FIG. 1. In FIG. 1, two target hybridization oligonucleotides are hybridized to a target RNA in the cell. Together, the two target hybridization oligonucelotides provide a hybrization site to which an amplification oligonucleotide can bind. Hybridization and/or subsequent washing of the amplification oligonucleotide may be performed at a temperature that allows hybridization to two proximal target hybridization oligonucleotides, but is above the melting temperature of the hybridization of the amplification oligonucleotide to just one target hybridization oligonucleotide. The first amplification oligonucleotide shown in FIG. 1 provides multiple hybridization sites, to which second amplification oligonucleotides can be bound, forming a branched pattern. Element-tagged oligonucleotides are bound multiple hybridization sites provided by the second amplification nucleotides shown in FIG. 1. Together, these amplification oligonucelotides (with or without element-tagged oligonucleotides) are referred to herein as a “multimer”.

In certain embodiments, hybridization may include contacting cells with one or more oligonucleotides, such as target hybridization oligonucleotides, amplification oligonucleotides, and/or mass-tagged oligonucleotides, and providing conditions under which hybridization can occur. Hybridization may be performed in a buffered solution, such as saline sodium-citrate (SCC) buffer, phosphate-buffered saline (PBS), saline-sodium phosphate-EDTA (SSPE) buffer, TNT buffer (having Tris-HCl, sodium chloride and Tween 20), or any other suitable buffer. Hybridization may be performed at a temperature around or below the melting temperature of the hybridization of the one or more oligonucleotides.

Specificity may be improved by performing one or more washes following hybridization, so as to remove unbound oligonucleotide. Increased stringency of the wash may improve specificity, but decrease overall signal. The stringency of a wash may be increased by increasing or decreasing the concentration of the wash buffer, increasing temperature, and/or increasing the duration of the wash. RNAse inhibitor may be used in any or all hybridization incubations and subsequent washes.

A first set of hybridization probes, including one or more target hybridizing oligonucleotides, amplification oligonucleotides and/or element-tagged oligonucleotides, may be used to label a first target nucleic acid. Additional sets of hybridization probes may be used to label additional target nucleic acids. Each set of hybridization probes may be specific for a different target nucleic acid. The additional sets of hybridization probes may be designed, hybridized and washed so as to reduce or prevent hybridization between oligonucleotides of different sets. In addition, the element-tagged oligonucleotide of each set may provide a unique signal. As such, multiple sets of oligonucleotides may be used to detect 2, 3, 5, 10, 15, 20 or more distinct nucleic acid targets.

### Use of Element-Tagged Oligonucleotides as Label Probes in RNAscope

A powerful in situ hybridization method called RNAscope® was recently developed by Advanced Cell Diagnostics, Inc. and described in U.S. Pat. No. 7,709,198 and U.S. Pub. No. 2012/0100540, each of which incorporated herein by reference in its entirety. In certain aspects, certain steps of hybridization, including target RNA binding, amplification, element-tagged oligonucleotide hybridization, and any related wash steps, may be similar to those described in the above incorporated patent and publication. As used below, the term “label probe” is interchangeable with “element-tagged oligonucleotide”, the term “capture probes” is interchangeable with “target hybridization oligonucleotides” and the term “pre-amplifier” and “amplifier” relate to the “amplification oligonucleotides” discussed above.

RNAscope® allows for direct visualization of RNA in situ. This method utilizes the oligonucleotide probe sets and novel signal amplification systems described below. The assay can be used on a variety of sample types including cultured cells, peripheral blood mononuclear cells (PBMCs), frozen tissue, and formalin-fixed paraffin embedded (FFPE) tissue. As demonstrated herein, the label probes may be element-tagged oligonucleotides instead of the fluorophore-labeled probes described in the above incorporated patent and publication.

The RNAscope® assay technology provides multiplex nucleic acid assays in single cells. At the core of this technology is the “double Z” oligonucleotide probe design, which allows robust amplification of specific hybridization signals without also amplifying nonspecific events. Each capture probe (“Z”) has a target-specific sequence which binds to the target mRNA, a spacer, and a “tail” sequence. Two capture probes (double Z) hybridize contiguously onto a target mRNA, and the two “tail” sequences form a 28-base hybridization site for the preamplifier. The double Z probe design ensures high fidelity of signal amplification because: 1) it is highly unlikely that a pair of target probes will hybridize nonspecifically juxtaposed to each other to form a binding site for the preamplifier; and 2) neither tail alone can bind efficiently to the preamplifier under the assay conditions. The preamplifier, amplifier and label probe are hybridized sequentially to each capture probe pair, resulting in the accumulation of as many as 8,000 label molecules per 1 kb of target RNA. The label probe can be conjugated to either an element tag, such as a polymer having multiple chelated metal ions, enabling viewing of hybridization signals by ionizing the sample and measuring presence of the element tag by mass spectrometry.

In addition, multiple signal amplifiers have been built that each recognizes a unique tail sequence on the target probes, allowing for the simultaneous visualization of multiple target RNAs. Importantly, this assay is compatible with partially degraded RNA present in archival FFPE tissues, since the double Z probe pairs target short regions of around 50 nucleotides or less in length.

In one example, RNAscope® was used to detect a target nucleic acid. In the method, a sample comprising one or more cell is provided. The cell tested comprises, or is suspected of comprising, the target nucleic acid. Provided in the assay are: a set of capture probes comprising two or more capture probes, a label probe comprising a label, and optionally preamplifiers and amplifiers.

In the method, the set of capture probes is hybridized, in the cell, to the target nucleic acid. The label probe is captured to the set of capture probes, thereby capturing the label probe to the target nucleic acid. The signal from the label is then detected. Since the label is associated with the target nucleic acid through the capture probes, presence of the label(s) in the cell indicates the presence of the corresponding nucleic acid target(s) in the cell. The methods are optionally quantitative. Thus, an intensity of the signal can be measured, and the intensity of the signal can be correlated with a quantity of the target nucleic acid in the cell. As another example, a signal spot can be counted for each copy of the target nucleic acid in order to quantify them.

In one aspect, the label probes bind directly to the capture probes. In another aspect, the label probes are captured to the capture probes indirectly, for example, through binding of preamplifiers and/or amplifiers. Use of amplifiers and preamplifiers can be advantageous in increasing signal strength, since they can facilitate binding of large numbers of label probes to each nucleic acid target.

Although both direct capture approach and indirect capture approach can be used in the instant technology, the indirect capture approach may be preferred because it enables the label probe to be target-independent and further disclosure will show that it can offer better specificity and sensitivity.

The capture probe is specially designed in the RNAscope® assay. In each capture probe, there is at least one section, T, complementary to a section on the target molecule, and another section. L, complementary to a section on the label probe. The T and L sections are connected by a section C. In a indirect capture embodiment, two adjacent capture probes are incorporated in a probe set targeting a gene of interest. T1 and T2 are designed to be complementary to two unique and adjacent sections on the target nucleic acid. L1 and L2, which can be different or the same, are complementary to two adjacent sections on the label probe. Their binding sections, T, L or both, are designed so that the linkage between the label probe and the target is unstable and tends to fall off at hybridization temperature when only one of the capture probes is in place. Such a design should enable exceptional specificity because the signal-generating label probe can only be attached to the target gene of interest when two independent capture probes both recognize the target and bind to the adjacent sequences or in very close proximity of the target gene. In one embodiment, the melting temperature, Tm, of the T sections of the two capture probes are designed to be significantly above the hybridization temperature while the Tm of the L sections is below the hybridization temperature. As a result, T sections bind to the target molecule strongly and stably duringhybridization, while L sections bind to the label probe weakly and unstably if only one of the capture probes is present. However, if both capture probes are present, the combination of L1 and L2 holds the label probe strongly and stably duringhybridization. For example, the T sections can be 20-30 nucleotides in length while the L sections are 13-15 nucleotides in length; C can be 0 to 10 nucleotides in length, e.g., 5 nucleotides. In another embodiment, Tm of the T sections is below hybridization temperature while Tm of the L sections is substantially above. In the same way, the linkage between the label probe and the target can only survive the hybridization when both capture probes are hybridized to the target in a cooperative fashion.

In the above classes of embodiments, the capture probes preferably hybridize to nonoverlapping polynucleotide sequences in their respective nucleic acid target. The capture probes can, but need not, cover a contiguous region of the nucleic acid target. Blocking probes, polynucleotides which hybridize to regions of the nucleic acid target not occupied by target probes, are optionally provided and hybridized to the target. For a given nucleic acid target, the corresponding capture probes and blocking probes are preferably complementary to physically distinct, nonoverlapping sequences in the nucleic acid target, which nonoverlapping sequences are preferably, but not necessarily, contiguous. Having the capture probes and optional blocking probes be contiguous with each other can in some embodiments enhance hybridization strength, remove secondary structure, and ensure more consistent and reproducible signal.

In detection of nucleic acid targets in a cell, the cell is typically fixed and permeabilized before hybridization of the capture probes, to retain the nucleic acid targets in the cell and to permit the capture probes, label probes, etc. to enter the cell. The cell is optionally washed to remove materials not captured to one of the nucleic acid targets. The cell can be washed after any of various steps, for example, after hybridization of the capture probes to the nucleic acid targets to remove unbound capture probes, after hybridization of the preamplifiers, amplifiers, and/or label probes to the capture probes, and/or the like.

In some embodiments, the cell is in suspension for all or most of the steps of the method, for ease of handling. However, the methods are also applicable to cells in solid tissue samples (e.g., tissue sections) and/or cells immobilized on a substrate (e.g., a slide or other surface). Thus, in one class of embodiments, the cell is in suspension in the sample comprising the cell, and/or the cell is in suspension during the hybridizing, capturing, and/or detecting steps. For example, the cell can be in suspension in the sample and during the hybridization, capture, optional washing, and detection steps. In other embodiments, the cell is in suspension in the sample comprising the cell, and the cell is fixed on a substrate during the hybridizing, capturing, and/or detecting steps. For example, the cell can be in suspension during the hybridization, capture, and optional washing steps and immobilized on a substrate during the detection step. In other embodiments, the sample comprises a tissue section.

### RNAscope Using Element-Tagged Oligonucleotides

A powerful in-situ hybridization method called RNAscope® was recently developed by Advanced Cell Diagnostics, Inc. and described in U.S. Pat. No. 7,709,198 and U.S. Pub. No. 2012/0100540, each of which incorporated herein by reference in its entirety. In certain embodiments, certain steps of hybridization in the subject invention, including target RNA binding, amplification, and any related wash steps, may be similar to those described in the above incorporated patent and publication.

RNAscope® allows for direct visualization of RNA in situ. This method utilizes the oligonucleotide probe sets and novel signal amplification systems described below. The assay can be used on a variety of sample types including cultured cells, peripheral blood mononuclear cells (PBMCs), frozen tissue, and formalin-fixed paraffin embedded (FFPE) tissue. As demonstrated herein, the label probes may be element-tagged oligonucleotides instead of the fluorophore-labeled probes described in the above incorporated patent and publication.

The RNAscope® assay technology provides multiplex nucleic acid assays in single cells. At the core of this technology is the “double Z” oligonucleotide probe design, which allows robust amplification of specific hybridization signals without also amplifying nonspecific events. Each capture probe (“Z”) has a target-specific sequence which binds to the target mRNA, a spacer, and a “tail” sequence. Two capture probes (double Z) hybridize contiguously onto a target mRNA, and the two “tail” sequences form a 28-base hybridization site for the preamplifier. The double Z probe design ensures high fidelity of signal amplification because: 1) it is highly unlikely that a pair of target probes will hybridize nonspecifically juxtaposed to each other to form a binding site for the preamplifier; and 2) neither tail alone can bind efficiently to the preamplifier under the assay conditions. The preamplifier, amplifier and label probe are hybridized sequentially to each capture probe pair, resulting in the accumulation of as many as 8,000 label molecules per 1 kb of target RNA. The label probe can be conjugated to either an element tag, such as a polymer having multiple chelated metal ions, enabling viewing of hybridization signals by ionizing the sample and measuring presence of the element tag by mass spectrometry.

In addition, multiple signal amplifiers have been built that each recognizes a unique tail sequence on the target probes, allowing for the simultaneous visualization of multiple target RNAs. Importantly, this assay is compatible with partially degraded RNA present in archival FFPE tissues, since the double Z probe pairs target short regions of around 50 nucleotides or less in length.

In one example, RNAscope® was used to detect a target nucleic acid. In the method, a sample comprising one or more cell is provided. The cell tested comprises, or is suspected of comprising, the target nucleic acid. Provided in the assay are: a set of capture probes comprising two or more capture probes, a label probe comprising a label, and optionally preamplifiers and amplifiers.

In the method, the set of capture probes is hybridized, in the cell, to the target nucleic acid. The label probe is captured to the set of capture probes, thereby capturing the label probe to the target nucleic acid. The signal from the label is then detected. Since the label is associated with the target nucleic acid through the capture probes, presence of the label(s) in the cell indicates the presence of the corresponding nucleic acid target(s) in the cell. The methods are optionally quantitative. Thus, an intensity of the signal can be measured, and the intensity of the signal can be correlated with a quantity of the target nucleic acid in the cell. As another example, a signal spot can be counted for each copy of the target nucleic acid in order to quantify them.

In one aspect, the label probes bind directly to the capture probes. In another aspect, the label probes are captured to the capture probes indirectly, for example, through binding of preamplifiers and/or amplifiers. Use of amplifiers and preamplifiers can be advantageous in increasing signal strength, since they can facilitate binding of large numbers of label probes to each nucleic acid target.

Although both direct capture approach and indirect capture approach can be used in the instant technology, the indirect capture approach may be preferred because it enables the label probe to be target-independent and further disclosure will show that it can offer better specificity and sensitivity.

The capture probe is specially designed in the RNAscope® assay. In each capture probe, there is at least one section, T, complementary to a section on the target molecule, and another section. L, complementary to a section on the label probe. The T and L sections are connected by a section C. In a indirect capture embodiment, two adjacent capture probes are incorporated in a probe set targeting a gene of interest. T1 and T2 are designed to be complementary to two unique and adjacent sections on the target nucleic acid. L1 and L2, which can be different or the same, are complementary to two adjacent sections on the label probe. Their binding sections, T, L or both, are designed so that the linkage between the label probe and the target is unstable and tends to fall off at hybridization temperature when only one of the capture probes is in place. Such a design should enable exceptional specificity because the signal-generating label probe can only be attached to the target gene of interest when two independent capture probes both recognize the target and bind to the adjacent sequences or in very close proximity of the target gene. In one embodiment, the melting temperature, Tm, of the T sections of the two capture probes are designed to be significantly above the hybridization temperature while the Tm of the L sections is below the hybridization temperature. As a result, T sections bind to the target molecule strongly and stably duringhybridization, while L sections bind to the label probe weakly and unstably if only one of the capture probes is present. However, if both capture probes are present, the combination of L1 and L2 holds the label probe strongly and stably duringhybridization. For example, the T sections can be 20-30 nucleotides in length while the L sections are 13-15 nucleotides in length; C can be 0 to 10 nucleotides in length, e.g., 5 nucleotides. In another embodiment, Tm of the T sections is below hybridization temperature while Tm of the L sections is substantially above. In the same way, the linkage between the label probe and the target can only survive the hybridization when both capture probes are hybridized to the target in a cooperative fashion.

In the above classes of embodiments, the capture probes preferably hybridize to nonoverlapping polynucleotide sequences in their respective nucleic acid target. The capture probes can, but need not, cover a contiguous region of the nucleic acid target. Blocking probes, polynucleotides which hybridize to regions of the nucleic acid target not occupied by target probes, are optionally provided and hybridized to the target. For a given nucleic acid target, the corresponding capture probes and blocking probes are preferably complementary to physically distinct, nonoverlapping sequences in the nucleic acid target, which nonoverlapping sequences are preferably, but not necessarily, contiguous. Having the capture probes and optional blocking probes be contiguous with each other can in some embodiments enhance hybridization strength, remove secondary structure, and ensure more consistent and reproducible signal.

In detection of nucleic acid targets in a cell, the cell is typically fixed and permeabilized before hybridization of the capture probes, to retain the nucleic acid targets in the cell and to permit the capture probes, label probes, etc. to enter the cell. The cell is optionally washed to remove materials not captured to one of the nucleic acid targets. The cell can be washed after any of various steps, for example, after hybridization of the capture probes to the nucleic acid targets to remove unbound capture probes, after hybridization of the preamplifiers, amplifiers, and/or label probes to the capture probes, and/or the like.

In some embodiments, the cell is in suspension for all or most of the steps of the method, for ease of handling. However, the methods are also applicable to cells in solid tissue samples (e.g., tissue sections) and/or cells immobilized on a substrate (e.g., a slide or other surface). Thus, in one class of embodiments, the cell is in suspension in the sample comprising the cell, and/or the cell is in suspension during the hybridizing, capturing, and/or detecting steps. For example, the cell can be in suspension in the sample and during the hybridization, capture, optional washing, and detection steps. In other embodiments, the cell is in suspension in the sample comprising the cell, and the cell is fixed on a substrate during the hybridizing, capturing, and/or detecting steps. For example, the cell can be in suspension during the hybridization, capture, and optional washing steps and immobilized on a substrate during the detection step. In other embodiments, the sample comprises a tissue section.

One aspect of the invention provides multiplex nucleic acid assays. Thus, one general class of embodiments includes methods of detecting two or more target nucleic acids. In the section above, method for detecting a single target nucleic acid using RNAscope® has been described. In this section, method for detecting a single target nucleic acid using a combination of RNAscope® and general ISH signal amplification method will be described first.

In one embodiment of the invention, a method of detecting a single target nucleic acid is provided. In this method, a sample comprising or suspected of comprising the target nucleic acid is provided. Also provided are: a set of two or more capture probes wherein the capture probes are capable of binding to the target nucleic acid, signal generating multimers, and signal amplification probes.

The signal generating multimer comprises at least a label probe. In certain embodiments, the signal generating multimer comprises multiple identical label probes hybridized to an amplifier. The amplifier is capable of hybridizing to the label probe, and is also capable of hybridizing to the set of two or more capture probes. The signal generating multimer may comprises a label probe, an amplifier capable of hybridizing to the label probe, and a preamplifier capable of hybridizing to the amplifier and also capable of hybridizing to the set of two or more capture probes.

The above methods are also useful for multiplex detection of nucleic acids, including detection of 2, 3, 5, 10, 15, 20 or more distinct nucleic acid targets.

### Signal Amplification

Signal amplification allows for the detection of RNA in the cell, particularly for low copy number mRNA. Herein we discuss a number of approaches to increase the element signal of an element-tagged oligonucleotide bound to a target RNA.

Element tags may be a polymer having a plurality of metal atoms, as described above. Element tags with 10 or more, 20 or more, 50 or more, 100 or more, 200 or more, 500 or more, 1000 or more metal atoms provide an enhanced signal. The polymers described herein may be of compact shape, so as to minimize size of the element tag and allow washing of unbound element-tagged oligonucleotide. Alternatively, element tags with linkers or a linear polymer may allow for more element tagged oligonucleotides to be associated with a target nucleic acid.

In addition, hybridization schemes that amplify signal by providing a plurality of hybridization sites to which element tagged oligonucleotides can bind, also amplify signal.

One balancing factor for the combination of the ISH signal amplification methods is the size and number of the amplification oligonucleotides and the element tags. When one of the preamplifiers binds to sample nonspecifically, it will attract the same large number of amplifiers and label probes onto itself as it does when binding to a target nucleic acid. Furthermore, the number of label probes which are bound to the few nonspecific binding preamplifiers may not generate any observable signals. However, if the signal of these label probes is further boosted by the additional signal amplification by a subsequent general ISH signal amplification, the false positive signal will be high enough to be detected.

One approach to reduce such false positive signal is to reduce the size of preamplifier so that, firstly, it is less likely to be trapped in cellular matrix; secondly, even when it is bound nonspecifically in the sample, only a smaller number of amplifier molecules will be hybridized to it. As a result, the signal produced by the nonspecific binding will likely be too low to be detected even when the signal is boosted by the general ISH signal amplification. In one embodiment of this approach, one preamplifier is designed to bind to between 1 to 16 different amplification oligonucleotides. In one embodiment, one preamplifier is designed to bind to between 2 to 10 different amplification oligonucleotides. In another embodiment, one preamplifier is designed to bind to between 2 to 5 different amplification oligonucleotides.

Additionally, depending on the mass spectrometer used, element tags having metal isotopes of certain mass ranges may be detected with higher sensitivity. Additionally, mass spectrometers with multiple detectors may have one detector at a higher sensitivity that detects metal ions from element-tagged oligonucleotides.

### Protein-Specific Mass Labels

The conjugation chemistries described herein may be used to conjugate an elemental tag to a non-oligonucleotide affinity reagent that specifically binds a target protein. In certain embodiments, the affinity reagent is an antibody or a fragment thereof. The antibody may be a “Primary antibody”, i.e., an antibody that binds specifically to an antigen injected into an animal. They can be extracted from the animal or can be made by recombinant means. Alternatively, the antibody may be a “Secondary antibody”, i.e., those antibodies that bind specifically to primary antibodies. For example, primary antibodies can be used as the antigen injected into an animal of a different species, to generate secondary antibodies. For example, rabbit secondary anti-mouse antibodies can be made by immunizing a rabbit with mouse antibodies.

In certain embodiments, the elemental tag may be conjugated to the antibody by reacting a maleimide of the elemental tag with a thiol group on a cysteine of the antibody (e.g., at the Fc portion of the antibody), under reducing conditions. Methods of conjugating elemental tags to antibodies are discussed in US Pub. No. 20080003616, which is incorporated herein by reference.

The affinity reagent may specifically bind any protein target, including proteins present on the membrane, in the nucleus, in the cytosol. The affinity reagent may be state-specific, such as an antibody that only binds to the phosphorylated version of a signaling protein.

In certain aspects, multiple element-tagged affinity reagents may be used, each having a unique element tag and specifically binding a distinct protein. In certain embodiments, a methods or kit may have 2 or more, 5 or more, 10 or more, 20 or more, or 40 or more distinct element-tagged affinity reagents.

### Systems and Methods for Simultaneous Detection of RNA and Protein

Mass spectrometry may be used to analyze cells by simultaneously detecting elemental isotopes from element-tagged oligonucleotides bound to target RNA and element-tagged affinity reagents bound to target proteins. Simultaneous detection may be within a fraction of a second, such as within 100 ms, 10 ms, 1 ms, 100 μs, 10 μs, 1 μs, and so forth. Elemental isotopes previously bound to both RNA and protein may be ionized together and detected by mass spectrometry at the same time. Two, 5, 10, 20, 50 or more, elemental isotopes may be detected, e.g., for example and not limitation, 2 to about 100, about 5 to about 75, about 5 to about 50 or about 5 to about 40 isotopes. As such, the RNA associated elemental isotopes and protein associated elemental isotopes may be detected in the same mass spectra.

In certain embodiments, cells may be analyzed by mass cytometry. A mass cytometer suitable for the methods described herein includes a sample container having the cells labeled with a first element tag bound to a target RNA sequence and a second element tag bound to a target protein. The mass cytometer may further include a cell injector in fluid communication with the sample container, and a mass spectrometer in fluid communication with the cell injector, the mass spectrometer comprising an ionization source to generate groups of elemental ions from the plume and an ion detector to detect the groups of elemental ions. In certain aspects, the cell injector may further include a nebulizer. The nebulizer may be coupled to a spray chamber, configured to deliver a spray comprising cells of the slurry of cells to the ionization source. The nebulizer may disperse the cells in an inert gas, such as Argon or any other noble gas.

In certain embodiments, cells may be analyzed by imaging mass cytometery. An imaging mass cytometer (IMC) suitable for the methods described herein includes cells immobilized on a solid support, wherein the cells are labeled with a first element tag bound to a target RNA sequence and a second element tag bound to a target protein. The IMC system may further include a laser ablation system configured to generate a plume from a cell, or a portion of a cell, immobilized on the solid support, and a mass spectrometer coupled to the solid support for receiving the plume. The mass spectrometer may have an ionization source to generate groups of elemental ions from the plume and an ion detector to detect the groups of elemental ions. In certain aspects, the IMC system may further include a fluidics system coupled to the laser ablation system and the mass spectrometer, configured to deliver the plume to the ionization source. The fluidics system may be configured to deliver a carrier gas to the plume generated by the laser ablation system. The laser ablation system may configured to generate a laser spot size of less than 100 micrometers in diameter, less than 10 micrometers in diameter, or less than 1 micrometer in diameter. In certain aspects, the solid support is housed in a laser ablation cell.

For the mass cytometer or IMC system described in any of the above embodiments, the ionization source may be any hard ionization source known in the art, including electron impact (EI), electrospray ionization (ESI), and inductively coupled plasma (ICP). For example, the ionization source may be an ICP torch. Further, the ICP torch may heat the sample by passing a radio-frequency current passed through a coil surrounding the sample. In addition, a number of mass-spectrometers are suitable for the subject methods, including mass spectrometers using time-of-flight (TOF) mass analyzers, quadrupole mass analyzers, ion traps, and fourier transform ion cyclotron resonance. In certain embodiments, the mass spectrometer is a time-of-flight mass spectrometer. The cells in the mass cytometer or IMC system may be prepared according to any of the above described embodiments, and may have any of the above described elemental tags and elemental-tagged oligonucleotides and affinity reagents.

The above described systems may be used in the subject methods to simultaneously detect RNA and protein. In certain embodiments, a method of detection includes providing cells labeled with a first element tag bound to a target RNA sequence and a second element tag bound to a target protein. The method may further include ionizing a cell, or a portion thereof, to produce an ionized first and second element tag, and simultaneously detecting the ionized first and second element tag. The ionization of may be performed by an inductively coupled plasma (ICP) torch. The method may further comprise nebulizing the cells in suspension prior to ionizing. In certain embodiments, the ionized first and second element tag may be detected by time-of-flight mass spectrometry.

In certain embodiment of IMC analysis, the method further includes ablating a cell or a portion thereof by laser ablation to produce a laser ablation plume comprising the first and second mas tag, prior to ionization. The method may further include delivering the laser ablation plume to an inductively coupled plasma (ICP) torch to perform the ionization. Additional embodiments of the methods of detection, using the above systems, cells, element tags, mass-tagged oligonucelotides and affinity reagents, and hybridization schemes are within the scope of the subject invention.

Additional mass cytometers and IMC systems are known in the art, and are described in US Pub. No. US 2008/0046194, 2011/0024615, 2013/0181126, 2014/0120550, 2014/0121117, and 2014/0287953, which are incorporated herein by reference.

### Compositions and Kits

Aspects of the invention include compositions and kits for the detection of nucleic acids in the cell. Compositions may include the element-labeled oligonlucleotides of any of the above embodiments. In certain embodiments, a kit may include two or more element-labeled oligonucleotides that specifically bind different nucleic acids (e.g., different RNA) and have element tags with different metal atoms. Alternatively or in addition, a kit may have a set of hybridization oligonucleotides, as described herein. Kits may have a plurality of element-labeled oligonucleotides and/or sets of hybridization oligonucleotides, to allow for multiplexed assay. Kits may also have one or more element-tagged affinity reagents of any of the above embodiment, such as one or more element-tagged antibodies.

Kits may additionally include any buffers, fixation and permeabilization reagents, modulators, or other components described in the present application. Reagents may be included in mixture or separately, in packets, tubes, multi-well strips or plates, and so forth. Reagents may be provided in solution or dried-down. A kit may further include instructions for use.

### Applications

Information-encoding gene transcripts, messenger RNA or mRNA, constitute only 1-2% of the total cellular RNA pool. The majority is made up of various non-coding small RNA. In normal tissue, the bulk of mRNA (75%) consists of transcripts expressed on average between 5 and 50 copies per cell. Low abundance transcripts (less than 5 copies per cell) represent 25% of total mRNA mass. RNA in situ hybridization allows the detection of specific gene expression transcripts in tissue sections and single cells. Probe design and labeling strategy with metal tags are important considerations for increasing resolution and specific target detection.

The development of metal labeled RNA detection (transcriptomics) can be performed in the same cell in parallel with protein and/or metabolite identification using metal labeled antibodies (proteomics), lectins, small molecules, etc. A multi-omics approach would be extremely valuable in understanding fundamental biology and disease mechanisms. The massive amounts of quantitative data generated by the Multi-omics Mass Cytometry platform will require sophisticated software that automatically or semi-automatically performs analysis of single cells. This area of research and development should receive priority status.

Further development of high-dimensional analysis of single cells within tissue sections is achieved by coupling metal-tagged immunostaining and in situ hybridization with high-resolution laser ablation (Imaging Mass Cytometry). This method preserves spatial information regarding cell localization in complex tissue matrix, as well as cell-cell interactions.

Information-encoding gene transcripts, messenger RNA or mRNA, constitute only 1-2% of the total cellular RNA pool. The majority is made up of various non-coding small RNA. The term “small RNA” includes many types of RNA: ribosomal rRNA, transport tRNA, small nuclear snRNA, small nucleolar snoRNA, long non-coding IncRNA, endoplasmic reticulum recognition 7SL RNA, male germ line PIWI-associated piRNA and short interfering double-strand siRNA, any of which may be detected using the methods disclosed herein. A large group of about 6000 single-stranded microRNA (miRNA) has been identified in living organisms. They are typically 17-24 nucleotide (nt) long RNAs that are processed from longer endogenous hairpin transcripts. miRNA, siRNA and piRNA are of special interest due to their roles in essential regulation processes such as RNA silencing and modifications of endogenous genes, viruses and selfish genome elements.

Detection and quantitation of miRNA in samples collected from cancer patients are of significant interest, particularly because they are altered in all types of human cancer. About one-third of microRNA show substantial tissue specificity, while others may vary in expression level, but are not particularly tissue or cell type-specific. Some miRNAs appear to act as oncogenes by contributing to the transformed phenotype when expressed at elevated levels in cancers, while others act as tumor suppressors, genes whose deletion or mutation helps cells along the multi-step process of carcinogenesis. Thus, miRNA profiling represents new diagnostic and prognostic tools for cancer patients.

In normal tissue, the bulk of mRNA (75%) consists of transcripts expressed on average between 5 and 50 copies per cell. Low abundance transcripts (less than 5 copies per cell) represent 25% of total mRNA mass.

In normal tissue, the bulk of mRNA (75%) consists of transcripts expressed on average between 5 and 50 copies per cell. Low abundance transcripts (less than 5 copies per cell) represent 25% of total mRNA mass.

Of the many technologies that profile cellular RNAs, the most commonly used are cDNA cloning followed by sequencing, sequencing by synthesis (RNA-seq; [5]) and in situ hybridization-based detection. Many of these methods are indirect as they first require reverse transcription mediated conversion of RNA to corresponding cDNA, amplification and labelling of the cDNA population, introducing method-specific bias, making accurate measurement of relevant gene expression problematic.

Fluorescent RNA in situ hybridization is based on base pairing of a complementary strand of a nucleotide probe, labeled with a detectable fluorescent marker to the gene transcript of interest within a fixed cell. The choice of probe, preparation of sample, the composition of the hybridization solution and washing conditions are all used for performing an efficient hybridization with high signal-to-noise ratio. Current fluorescent-based in situ hybridization methods without amplification have a detection level of the order of 10-20 copies of mRNA per cell.

Recently, detection of low copy number RNA has been achieved in fluorescence based flow cytometry (Hanley et al., “Detection of low abundance RNA molecules in individual cells by flow cytometry. Supplement,” PLoS One, vol. 8, no. 2, 2013).

Mass Cytometry multi-omics approach will be useful, since each cell with its own milieu of active transcripts, proteins and metabolites would provide a complete physiological fingerprint of its processes, signaling pathways, and stress responses at a precise moment in time. The amount of biological noise is expected to be reduced and will improve data analysis within one platform.

“Single cell” multi-omics is directed at collecting information from a single cell, but it is not the same cell that is interrogated by each of the “omics” platforms, although the source of cell may be the same. Furthermore, all current high-throughput technologies use amplification chemistries to generate enough starting material from a single cell. The disadvantage of this approach lies in the propensity of different hardware and software platforms to introduce technical artifacts, which lead to data bias and erroneous sample differences in the absence of a biological cause. Moreover, lack of data standardization across different omics techniques interferes with cross-experiment comparisons, difficulties in assessing data-quality, context and lab-to-lab variations.

By combining Transcriptomics, Proteomics and Metabolomics using Mass Cytometry and high density data analysis, multi-omics can become truly dedicated to the analysis of a single cell.

### Examples

The following examples provide experimental data showing the production of metal-tagged oligonucleotides and their use in the simultaneous detection of both RNA and protein by mass cytometry.

5′ thiol-functionalized oligonucleotides were incubated in the presence of 25 mM TCEP, at room temperature for 2 hours. Element tags were prepared by separately incubating branched polymers (having multiple conjugation groups and functionalized with maleimide) in the presence of Ho165, Tm169, Eu153, or Yb176 at 37 degrees Celsius for 1 hour. Both the oligonucleotides and the element tags were separately purified by spin filtering. 0.06 mg of the 5′ thiol-functionalized oligonucleotides and 0.1 mg of the maleimide-functionalized element tags were combined and incubated at 37 degrees Celsius for 90 minutes in the presence of TCEP, then stored overnight.

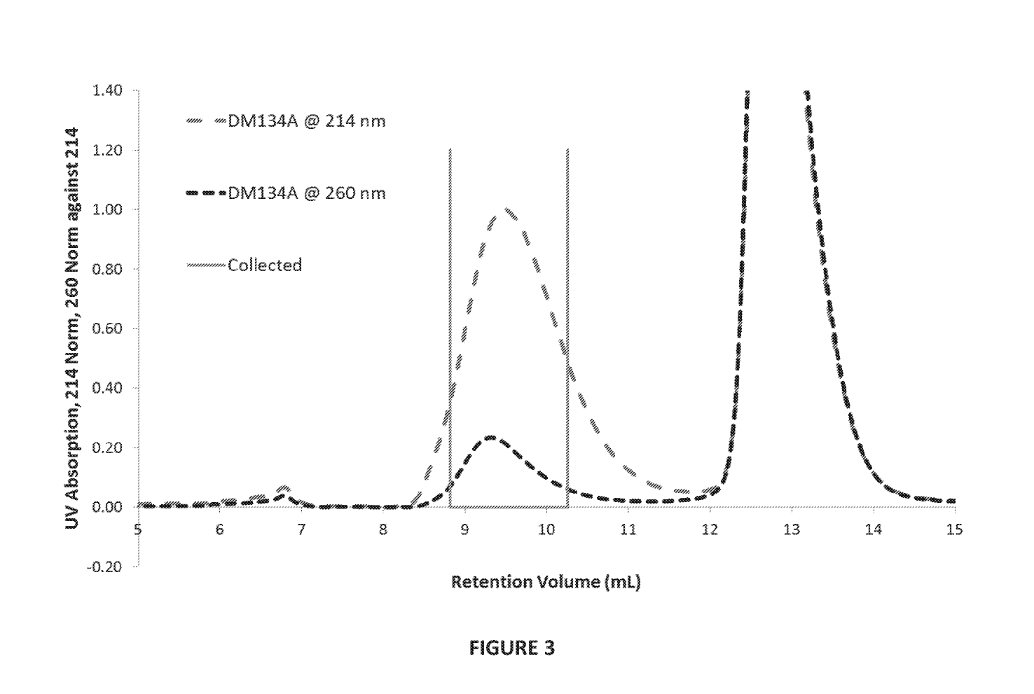

FIG. 3 shows a fast-protein liquid chromatography (FPLC) graph of a resulting element-labeled oligonucleotide (DM134A). The element-tag absorbs at 214 nm but not 260 nm, while the oligomer absorbs at 214 nm and 260 nm. The left peak (element-tagged oligonucleotide) shows a higher 214 nm to 260 nm absorbance ratio compared to the right peak, and was collected.

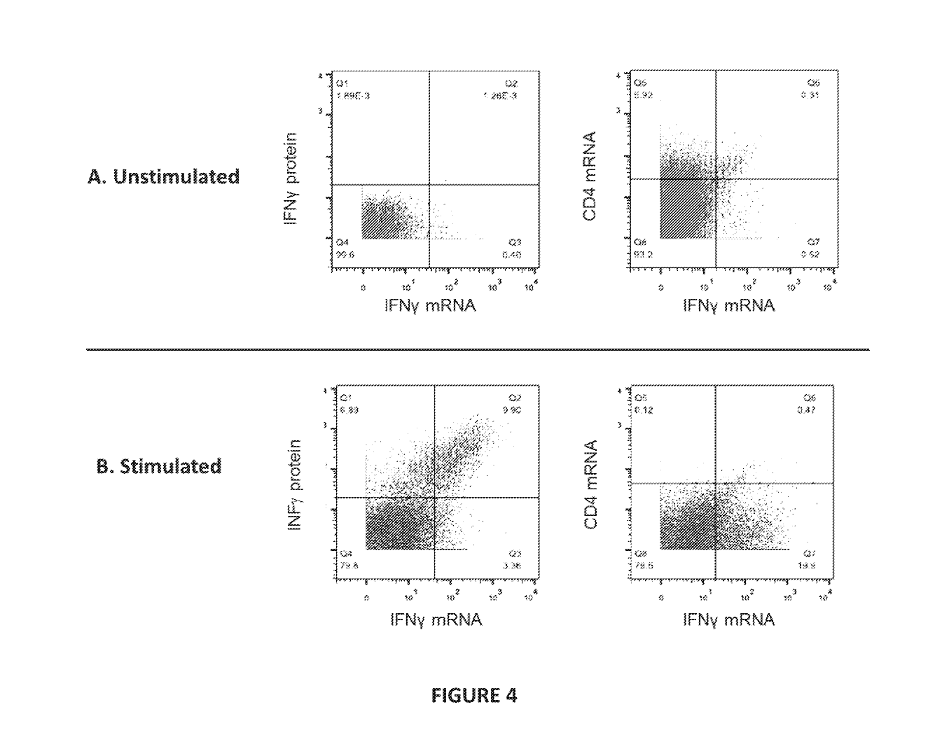

In FIG. 4, unstimulated human PBMCs, and PBMs stimulated with PMA (50 ng/mL) and ionomycin (1 ug/mL), were cultured for 5 hours in the presence of BD GolgiStop. Cells were washed, fixed and permeabilized. Cells were then stained for intracellular IFN gamma protein, IFN gamma mRNA (present in high copy number), and CD4 mRNA (present at low copy number). IFN gamma protein was stained using an anti-IFN gamma antibody conjugated to an Er168 elemental tag. The IFN gamma mRNA and CD4 mRNA was stained using separate sets of hybridization oligonucleotides, each having a distinct mass-tagged oligonucleotide. As seen in FIG. 4B, under stimulation, both IFN gamma mRNA and protein expression were detected and are highly correlated with one another.

What is claimed is:

1. A method of simultaneously detecting RNA and protein in cells by mass spectrometry, comprising:
a) providing cells labeled with a first element tag bound to a target RNA sequence and a second element tag bound to a target protein;
b) ionizing a cell, or a portion thereof, to produce an ionized first and second element tag; and
c) simultaneously detecting the ionized first and second element tag by mass spectrometry.

2. The method of claim 1, wherein the ionization of step b) is performed by an inductively coupled plasma (ICP) torch.

3. The method of claim 1 or 2, further comprising nebulizing the cells in suspension prior to step b) of ionizing.

4. The method of any of claims 1 to 3, wherein mass spectrometry of step c) is time-of-flight mass spectrometry.

5. The method of any of claims 1 to 4, further comprising ablating a cell or a portion thereof by laser ablation to produce a laser ablation plume comprising the first and second mass tag, prior to the ionization of step b).

6. The method of claim 5, further comprising delivering the laser ablation plume to an inductively coupled plasma (ICP) torch to perform the ionization of step b).

7. The method of any of claims 1 to 6, wherein the first and second element tags each comprise a different metal atom.

8. The method of claim 7, wherein the metal atoms of the first and second element tags are selected from a metalloid, rare earth, transition metal, or lanthanide.

9. The method of claim 7 or 8, wherein the first and second element tags each comprises a polymer comprising a plurality of identical metal atoms.

10. The method of claim 9, wherein the first and second element tags each comprises between 10 and 1000 metal atoms.

11. The method of claim 10, wherein the first and second element tags each comprises between 10 and 100 metal atoms.

12. The method of any of claims 9 to 11, wherein the first and second element tags each comprises a plurality of metal-binding pendant groups.

13. The method of claim 12, wherein the identical metal-binding pendant groups are selected from diethylenetriaminepentaacetate (DTPA) ligand, a 1,4,7,10-tetraazacyclododecane-1,4,7,10-tetraacetic acid (DOTA) ligand, and an amide or an ester of any of the forgoing.

14. The method of any of claims 9 to 13, wherein the polymer comprises N—N-dimethyl acrylamide and N-acryloxysuccinimide.

15. The method of any of claims 9 to 14, wherein the element tag is a branched polymer.

16. The method of any of claims 9 to 14, wherein the element tag is a linear polymer.

17. The method of any of claims 7 to 16, wherein the element tag comprises a C5-C20 non-cyclic alkyl spacer.

18. The method of any of claims 7 to 17, wherein the element tag comprises a polyelthylene glycol (PEG) spacer.

19. The method of any of claims 1 to 18, wherein the first element tag is an element-tagged oligonucleotide hybridized, directly or indirectly, to the target RNA sequence.

20. The method of any of claims 1 to 19, wherein the cells comprise at least 2 element tags that are each bound to a different target RNA sequence and that each comprise a different metal.

21. The method of any of claims 1 to 20, wherein the cells comprise at least 5 element tags that are each bound to a different target RNA sequence and that each comprise a different metal.

22. The method of any of claims 1 to 21, wherein the cells comprise at least 10 element tags that are each bound to a different target RNA sequence and that each comprise a different metal.

23. The method of any of claims 9 to 22, wherein the element tags are not distinguishable from each other by fluorescence.

24. The method of any of claims 1 to 23, wherein the second element tag is conjugated to an affinity reagent that specifically binds the target protein.

25. The method of claim 24, wherein the affinity reagent is an antibody or a fragment thereof.

26. The method of any of claims 1 to 25, wherein the cells are in a slurry.

27. The method of any of claims 1 to 26, wherein the cells are immobilized on a solid support.

28. The method of any of claims 1 to 27, wherein the cells are in a tissue section.

29. The method of any of claims 1 to 28, wherein the cells are peripheral blood mononuclear cells.

30. The method of any of claims 1 to 29, wherein the cells are primary cells.

31. The method of any of claims 1 to 30, wherein the cells are of an immortalized cell line.

32. A method of preparing a biological sample comprising cells for detection of RNA and protein by mass spectrometry, comprising:
a) fixing at least some of the cells of the biological sample;
b) permeabilizing at least some of the fixed cells;
c) hybridizing an element-tagged oligonucleotide, directly or indirectly, to a target RNA sequence present in at least some of the permeabilized cells; and
d) removing unbound element tagged oligonucleotide.

33. The method of claim 32, wherein step c) of hybridizing further comprises contacting the cells with two or more target hybridization oligonucleotides, and hybridizing the two or more target hybridization oligonucleotides to the target RNA.

34. The method of claim 33, wherein step c) of hybridizing further comprises contacting the cells with one or more amplification oligonucleotides, and hybridizing the one or more amplification oligonucleotides to the two or more target hybridization oligonucleotides and to multiple identical element-tagged oligonucleotides.

35. The method of claim 33, wherein step c) of hybridizing further comprises contacting the cells with a multimer comprising multiple identical element-tagged oligonucleotides, and hybridizing the multimer to both of the two or more target hybridization oligonucleotides.

36. The method of claim 32, wherein step c) of hybridizing further comprises contacting the cells with a set of hybridization oligonucleotides, capable of hybridizing to both the target RNA sequence and to the element-tagged oligonucleotide.

37. The method of claim 36, wherein the set of hybridization oligonucleotides comprises at least two target hybridization oligonucleotides, each capable of hybridizing to a different sub-sequence of the target RNA.

38. The method of claim 37, wherein each target hybridization oligonucleotide comprises a sequence of between 10 and 40 nucleotides long that is complementary to a different sub-sequence of the target RNA.

39. The method of claim 38, wherein each target hybridization oligonucleotide comprises a sequence of between 15 and 30 nucleotides long that is complementary to a different sub-sequence of the target RNA.

40. The method of any of claims 38 to 39, wherein each target hybridization oligonucleotide is between 20 and 80 nucleotides long.

41. The method of claim 40, wherein each target hybridization oligonucleotide is between 30 and 50 nucleotides long.

42. The method of any of claims 38 to 41, wherein the different sub-sequences are proximal to one another.

43. The method of any of claims 36 to 42, wherein the set of hybridization oligonucleotides further comprises an amplification oligonucleotide, capable of hybridizing to the at least two target hybridization oligonucleotides and providing a plurality of identical hybridization sites.

44. The method of claim 43, wherein the amplification oligonucleotide comprises a plurality of proximal 8-20 nucleotide long sequences, each complementary to a sub-sequence of a different target hybridization oligonucleotide.

45. The method of claim 43 or 44, wherein each hybridization site is between 8 and 30 base pairs long.

46. The method of claim 45, wherein each hybridization site is between 10 and 20 base pairs long.

47. The method of any of claims 43 to 46, wherein the amplification oligonucleotide between 40 and 400 nucleotides long.

48. The method of any of claims 43 to 46, wherein the identical hybridization sites are capable of hybridizing the element tagged oligonucleotide.

49. The method of any of claims 36 to 48, wherein the set of hybridization oligonucleotides further comprises one or more additional amplification oligonucleotides, each capable of binding to a single hybridization site and providing a plurality of additional hybridization sites, wherein at least some of the additional hybridization sites are capable of hybridizing to the element tagged oligonucleotide.

50. The method of any of claims 32 to 49, wherein the element tagged oligonucleotide comprises an element tag linked to the oligonucleotide at the 5′ position of the 5′ nucleotide.

51. The method of any of claims 32 to 49, wherein the element tagged oligonucleotide comprises an element tag linked to the oligonucleotide at the 3′ position of the 3′ nucleotide.

52. The method of any of claims 32 to 51, wherein the element tag is conjugated to the oligonucleotide by a biotin bound to an avidin or avidin homologue.

53. The method of any of claims 32 to 51, wherein the element tag is conjugated to the oligonucleotide by an amine-to-sulflhydryl crosslinker.

54. The method of any of claims 32 to 51, wherein the element tag is conjugated to the oligonucleotide by a thioether bond.

55. The method of any of claims 32 to 51, wherein the element tag is conjugated to the oligonucleotide by a secondary amine and a sulfide.

56. The method of any of claims 32 to 51, wherein the element tag is conjugated to the oligonucleotide by a phsophoramidate bond.

57. The method of any of claims 32 to 51, wherein the element tag is conjugated to the oligonucleotide by an amide bond.

58. The method of any of claims 32 to 57, wherein the element tag is separated from the oligonucleotide by a polyethylene glycol (PEG) spacer.

59. The method of any of claims 32 to 57, wherein the element tag is separated from the oligonucleotide by a C5-C20 non-cyclic alkyl spacer.

60. The method of any of claims 32 to 59, wherein the element tagged oligonucleotide is between 8 and 50 nucleotides long.

61. The method claim 60, wherein the element tagged oligonucleotide is between 12 and 30 nucleotides long.

62. The method of any of claims 32 to 61, further comprising contacting the biological sample with an element tagged affinity reagent, before or after cells are permeabilized in step b), wherein the element tagged affinity reagent specifically binds to a target protein present on or in the cell.

63. The method of any of claims 32 to 62, further comprising culturing the cells, or a subset thereof, in the presence of one or more modulators prior to step a) of fixing the cells.

64. The method of claim 63, wherein the one or more modulators include PMA and/or ionomycin at a concentration effective to stimulate the cells.

65. The method of claim 63 or 64, further comprising culturing the cells in the presence of a golgi plug prior to step a) of fixing the cells.

66. A method of preparing an element tagged oligonucleotide, comprising:
a) providing an oligionucleotide comprising a first reactive group;
b) contacting the oligonucleotide with an element tag comprising a second reactive group; and
c) reacting the first and second reactive groups so as to form a covalent bond linking the oligonucleotide to the element tag.

67. The method of claim 66, wherein the first functional group is on the 5′ position of the 5′ nucleotide.

68. The method of claim 66, wherein the first functional group is on the 3′ position of the 3′ nucleotide.

69. The method of any of claims 66 to 68, wherein one of the first and second reactive groups is thiol and the other of the first and second reactive groups is reactive with thiol.

70. The method of claim 69, wherein the first reactive group is a thiol and the second reactive group is a maleimide.

71. The method of any of claims 66 to 70, wherein step c) of reacting the first and second reactive groups is performed under reducing conditions.

72. The method of claim 71, wherein step c) of reacting the first and second reactive groups is performed in the presence of 3,3′,3″-Phosphanetriyltripropanoic acid (TCEP).

73. The method of any of claims 66 to 68, wherein one of the first and second reactive groups is an amine and the other of the first and second reactive groups is reactive with amine.

74. The method of any of claims 66 to 68, wherein the first reactive group is a 5′ phosphate.

75. The method of claim 74, wherein the second reactive group is an amine.

76. The method of claim 75, wherein step c) comprises reacting the 5′ phosphate with the amine in the presence of 1-Ethyl-3-[3-dimethylaminopropyl]carbodiimide (EDC) and 1,3-diazacyclopenta-2,4-diene (imidazole).

77. The method of any of claims 66 to 68, wherein one of the first and second reactive groups is a haloacetyl and the other of the first and second reactive groups is a thiol.

78. The method of claim 77, wherein the haloacetyl comprises bromine, chlorine, or iodine.

79. The method of any of claims 66 to 68, wherein step c) comprises reacting the first and second reactive groups with a linker molecule.

80. The method of any of claims 66 to 68, wherein the linker molecule comprises a C5-C20 non-cyclic alkyl spacer.

81. The method of claim 80, wherein the linker molecule comprises polyelthylene glycol (PEG) spacer

82. The method of claim 81, wherein the linker molecule is an amine-to-sulfhydryl crosslinker.

83. The method of any of claims 66 to 68, wherein the linker molecule comprises a thioester and a primary amine.

84. The method of any of claims 66 to 83, wherein the ratio of the element tag molecules to oligonucleotide molecules is less than 0.9.

85. The method of any of claims 66 to 84, wherein the element tag comprises a metal atom.

86. The method of claim 85, wherein the metal atom is a metalloid, rare earth, transition metal, or lanthanide.

87. The method of any of claims 66 to 84, wherein the element tag comprises a polymer comprising a plurality of identical metal atoms.

88. The method of claim 87, wherein the element tag is a polymer comprising a plurality of metal-binding pendant groups.

89. The method of claim 88, wherein the identical metal-binding pendant groups are selected from diethylenetriaminepentaacetate (DTPA) ligand, a 1,4,7,10-tetraazacyclododecane-1,4,7,10-tetraacetic acid (DOTA) ligand, and an amide or an ester of any of the forgoing.

90. The method of any of claims 87 to 89, wherein the polymer comprises N—N-dimethyl acrylamide and N-acryloxysuccinimide.

91. The method of any of claims 87 to 90, wherein the element tag is a branched polymer.

92. The method of any of claims 87 to 90, wherein the element tag is a linear polymer.

93. The method of any of claims 87 to 92, wherein the element tag comprises between 10 and 100 metal atoms.

94. The method of any of claims 66 to 93, wherein the element tag is not detectable by fluorescence.

95. The method of any of claims 87 to 94, wherein the element tag comprises a C5-C20 non-cyclic alkyl spacer.

96. The method of any of claims 87 to 94, wherein the element tag comprises a polyelthylene glycol (PEG) spacer.

97. A system for simultaneous analysis of RNA and protein in a slurry of cells, comprising:
a) a sample container comprising a slurry of cells labeled with a first element tag bound to a target RNA sequence and a second element tag bound to a target protein;
b) a cell injector in fluid communication with the sample container;
c) a mass spectrometer in fluid communication with the cell injector, the mass spectrometer comprising an ionization source to generate groups of elemental ions from the plume and an ion detector to detect the groups of elemental ions.

98. The system of claim 97, wherein the cell injector comprises a nebulizer.

99. The system of claim 98, wherein the nebulizer is coupled to a spray chamber, configured to deliver a spray comprising cells of the slurry of cells to the ionization source.

100. A system for simultaneous analysis of RNA and protein in immobilized cells, comprising:
a) cells immobilized on a solid support, wherein the cells are labeled with a first element tag bound to a target RNA sequence and a second element tag bound to a target protein;
b) a laser ablation system configured to generate a plume from a cell, or a portion of a cell, immobilized on the solid support; and
c) a mass spectrometer coupled to the solid support for receiving the plume, the mass spectrometer having an ionization source to generate groups of elemental ions from the plume and an ion detector to detect the groups of elemental ions.

101. The system of claim 100, further comprising a fluidics system coupled to the laser ablation system and the mass spectrometer, configured to deliver the plume to the ionization source.

102. The system of claim 101, wherein the fluidics system is configured to deliver a carrier gas to the plume generated by the laser ablation system.

103. The system of any of claims 100 to 102, wherein the laser ablation system is configured to generate a laser spot size of less than 10 micrometers in diameter.

104. The system of any of claims 100 to 103, wherein the laser ablation system is configured to generate a laser spot size of less than 1 micrometer in diameter.

105. The system of any of claims 100 to 104, wherein the solid support is housed in a laser ablation cell.

106. The system of any of claims 97 to 105, wherein the ionization source is an inductively coupled plasma (ICP) torch.

107. The system of any of claims 97 to 106, wherein the mass spectrometer is a time-of-flight mass detector.

108. The system of any of claims 97 to 107, wherein the first and second element tags each comprise a different metal atom.

109. The system of any of claims 97 to 108, wherein the metal atoms of the first and second element tags are selected from a metalloid, rare earth, transition metal, or lanthanide.

110. The system of any of claims 97 to 109, wherein the first and second element tags each comprises a polymer comprising a plurality of identical metal atoms.

111. The system of claim 110, wherein the first and second element tags each comprises between 10 and 1000 metal atoms.

112. The system of claim 111, wherein the first and second element tags each comprises between 10 and 100 metal atoms.

113. The system of any of claims 110 to 112, wherein the first and second element tags each comprises a plurality of metal-binding pendant groups.

114. The system claim 113, wherein the identical metal-binding pendant groups are selected from diethylenetriaminepentaacetate (DTPA) ligand, a 1,4,7,10-tetraazacyclododecane-1,4,7,10-tetraacetic acid (DOTA) ligand, and an amide or an ester of any of the forgoing.

115. The system of any of claims 110 to 114, wherein the polymer comprises N—N-dimethyl acrylamide and N-acryloxysuccinimide.

116. The system of any of claims 110 to 115, wherein the element tag is a branched polymer.

117. The system of any of claims 97 to 116, wherein the first and the second element tags are not distinguishable from each other by fluorescence.

118. The system of any of claims 97 to 117, wherein the element tag comprises a C5-C20 non-cyclic alkyl spacer.

119. The system of any of claims 97 to 117, wherein the element tag comprises a polyelthylene glycol (PEG) spacer.

120. The system of any of claims 97 to 119, wherein the first element tag is an element-tagged oligonucleotide hybridized, directly or indirectly, to the target RNA sequence.

121. The system of any of claims 97 to 120, wherein the cells comprise at least 2 element tags that are each bound to a different target RNA sequence.

122. The system of any of claim 121, wherein the cells comprise at least 5 element tags that are each bound to a different target RNA sequence.

123. The system of claim 122, wherein the cells comprise at least 10 element tags that are each bound to a different target RNA sequence.

124. The system of any of claims 97 to 123, wherein the second element tag is conjugated to an affinity reagent that specifically binds the target protein.

125. The system of claim 124, wherein the affinity reagent is an antibody or a fragment thereof.

126. The system of any of claims 97 to 125, wherein the cells are primary cells.

127. The system of claim 126, wherein the cells are peripheral blood mononuclear cells.

128. The system of any of claims 97 to 125, wherein the cells are of an immortalized cell line.

129. The system of any of claims 100 to 105, wherein the cells are in a tissue section.

130. A composition comprising an element-tagged oligonucleotide, wherein the element tag comprises a plurality of identical metal atoms.

131. The composition of claim 130, wherein the element tagged oligonucleotide comprises an element tag linked to the oligonucleotide at the 5′ position of the 5′ nucleotide.

132. The composition of claim 130, wherein the element tagged oligonucleotide comprises an element tag linked to the oligonucleotide at the 3′ position of the 3′ nucleotide.

133. The composition of any of claims 130 to 132, wherein the element tag is conjugated to the oligonucleotide by a biotin bound to an avidin or avidin homologue.

134. The composition of any of claims 130 to 132, wherein the element tag is conjugated to the oligonucleotide by an amine-to-sulflhydryl crosslinker.

135. The composition of any of claims 130 to 132, wherein the element tag is conjugated to the oligonucleotide by a thioether bond.

136. The composition of any of claims 130 to 132, wherein the element tag is conjugated to the oligonucleotide by a secondary amine and a sulfide.

137. The composition of any of claims 130 to 132, wherein the element tag is conjugated to the oligonucleotide by a phosphoramidate bond.

138. The composition of any of claims 130 to 132, wherein the element tag is conjugated to the oligonucleotide by an amide bond.

139. The composition of any of claims 130 to 138, wherein the element tag is separated from the oligonucleotide by a polyethylene glycol (PEG) spacer.

140. The composition of any of claims 130 to 138, wherein the element tag is separated from the oligonucleotide by a C5-C20 non-cyclic alkyl spacer.

141. The composition of any of claims 130 to 140, wherein the element tagged oligonucleotide is between 8 and 50 nucleotides long.

142. The composition of claim 141, wherein the element tagged oligonucleotide is between 12 and 30 nucleotides long.

143. The composition of any of claims 130 to 142, wherein the element tag comprises a metal atom.

144. The composition of claim 143, wherein the metal atom is a metalloid, rare earth, transition metal, or lanthanide.

145. The composition of claim 144, wherein the element tag comprises a polymer comprising a plurality of identical metal atoms.

146. The composition of claim 145, wherein the element tag is a polymer comprising a plurality of metal-binding pendant groups.

147. The composition of claim 146, wherein the identical metal-binding pendant groups are selected from diethylenetriaminepentaacetate (DTPA) ligand, a 1,4,7,10-tetraazacyclododecane-1,4,7,10-tetraacetic acid (DOTA) ligand, and an amide or an ester of any of the forgoing.

148. The composition of any of claim 145 or 147, wherein the polymer comprises N—N-dimethyl acrylamide and N-acryloxysuccinimide.

149. The composition of any of claim 145 or 148, wherein the element tag is a branched polymer.

150. The composition of any of claim 145 or 148, wherein the element tag is a linear polymer.

151. The composition of any of claim 145 or 150, wherein the element tag comprises between 10 and 1000 metal atoms.

152. The composition of claim 151, wherein the element tag comprises between 10 and 1000 metal atoms.

153. The composition of any of claim 145 or 150, wherein the element tag is not detectable by fluorescence.

154. A kit comprising:
a) An element-tagged oligonucleotide according to any of claims 130 to 153; and
b) a second element tagged oligonucleotide;
wherein each element tagged oligonucleotide comprises a different metal and a different sequence.

155. The kit of claim 154, further comprising a plurality of distinct element tagged oligonucleotides, wherein each element tagged oligonucleotide comprises a different sequence and a different element tag.

156. The kit of claim 154 or 155, wherein the plurality of distinct element tagged oligonucleotides comprises at least 5 distinct element tagged oligonucleotides.

157. The kit of claim 156, wherein the plurality of distinct element tagged oligonucleotides comprises at least 10 distinct element tagged oligonucleotides.

158. The kit of claim 154 or 155, further comprising a third element tag conjugated to an affinity reagent that specifically binds a target protein.

159. The kit of claim 158, wherein the affinity reagent is an antibody or a fragment thereof.

160. A kit comprising:
a) an element-tagged oligonucleotide according to any of claims 130 to 153; and
b) a set of hybridization oligonucleotides capable of simultaneously:
i. hybridizing to a target RNA, and
ii. providing a plurality of identical hybridization sites each capable of hybridizing to the element-tagged oligonucleotide.

161. The kit of claim 160, wherein the set of hybridization oligonucleotides comprises at least two target hybridization oligonucleotides, each capable of hybridizing to a different sub-sequence of the target RNA.

162. The kit of claim 160 or 161, wherein each target hybridization oligonucleotide comprises a sequence of between 10 and 40 nucleotides long that is complementary to a different sub-sequence of the target RNA.

163. The kit of claim 162, wherein each target hybridization oligonucleotide comprises a sequence of between 15 and 30 nucleotides long that is complementary to a different sub-sequence of the target RNA.

164. The kit of any of claims 161 and 163, wherein each target hybridization oligonucleotide is between 20 and 80 nucleotides long.

165. The kit of claim 164, wherein each target hybridization oligonucleotide is between 30 and 50 nucleotides long.

166. The kit of any of claims 161 to 165, wherein the different sub-sequences on the target RNA are proximal to one another.

167. The kit of any of claims 161 to 166, wherein the set of hybridization oligonucleotides further comprises an amplification oligonucleotide, capable of hybridizing to the at least two target hybridization oligonucleotides and providing a plurality of identical hybridization sites complementary to at least a portion of the mass-tagged oligonucleotide.

168. The kit of any of claims 161 to 166, wherein the set of hybridization oligonucleotides further comprises a plurality of amplification oligonucleotides, together capable of hybridizing to the at least two target hybridization oligonucleotides and providing a plurality of identical hybridization sites complementary to at least a portion of the mass-tagged oligonucleotide.

169. The kit of any of claims 167 to 169, wherein each hybridization site is between 8 and 30 base pairs long.

170. The kit of claim 169, wherein each hybridization site is between 10 and 20 base pairs long.

171. The kit of any of claims 167 to 170, wherein each of the amplification oligonucleotides are between 40 and 400 nucleotides long.

172. The kit of any of claims 167 to 171, wherein the one or more amplification oligonucleotides and the mass-tagged oligonucleotide are provided together as a single multimer.
